(19) United States
(12) Patent Application Publication
(10) Pub. No.: US 2007/0054325 A1
(43) Pub. Date: Mar. 8, 2007

(54) Materials and methods for screening modulators of neural regeneration

(75) Inventors: Milos Pekny, Gothenburg (SE); Marcela Pekna, Gothenburg (SE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: REGLIA, Gothenburg (SE)

(21) Appl. No.: 11/581,207

(22) Filed: Oct. 13, 2006

Related U.S. Application Data
(63) Continuation-in-part of application No. PCT/EP05/03946, filed on Apr. 14, 2005.

(51) Int. Cl. G01N 33/567; G01N 33/53

(52) U.S. Cl. 435/7.2

(57) ABSTRACT

The invention provides materials and methods used to identify modulators of neurogenesis and neuroregeneration, and use of said modulators in the treatment of neurodegenerative diseases and neurological injury.

The present application is a continuation-in-part of PCT/EP2005/003946, which claims benefit under 35 U.S.C. § 119 of U.S. Patent Application No. 60/562,866, which was filed Apr. 15, 2004, each of which is incorporated herein by reference in its entirety.

### FIELD OF THE INVENTION

The present invention relates generally to material and methods used to identify modulators of neurogenesis and neuroregeneration, and use of said modulators in the treatment of neurodegenerative diseases and neurological injury.

### BACKGROUND

Astrocytes are the most abundant cells in the central nervous system (CNS). The importance of astrocytes in the maintenance of the homeostasis of the CNS, nutrition of neuronal cells and neurotransmitter recycling has been known for years. However, recently, astrocytes have been proposed to control both the number and the character of neuronal synapses. Little is known about function of astrocytes in CNS pathologies. In CNS trauma, tumor growth, brain ischemia or neurodegenerative diseases, astrocytes become reactive. Two hallmarks of this reaction, known as reactive gliosis (RG), are astroctye hypertrophy/prominent extension of cellular processes and upregulation of intermediate filaments (IFs), a part of the cytoskeleton, composed of three IF proteins—nestin, vimentin and GFAP. RG is accompanied by an altered expression profile of many genes (Eng et al., Brain Pathol. 4: 229-237, 1994). The importance of RG in CNS pathologies remains poorly understood. It has been speculated that this reaction may be both positive and negative. RG may contribute to the healing process; stimulate proliferation of neural stem cells and thereby increase neurogenesis; reconstitute a locally damaged blood-brain barrier; or form a barrier against the migration of tumor cells. Alternatively, reactive astrocytes may constitute an obstacle to neuroregeneration by functioning as a physical and/or chemical barrier, or produce cytokines and components of extracellular matrix which can stimulate tumor cell migration and/or proliferation.

In a striking contrast to the peripheral nervous system, the regenerative capacity of the CNS is extremely limited despite the fact that neural stem cells are present in the CNS throughout life. Both endogenous neural stem cells and neural implants, grafted to replace lost neurons, fail to form functional connections to the extent that would influence the clinical outcome in conditions such as CNS trauma, stroke, or neurodegenerative diseases. The reason for the highly restricted regenerative capacity of the mammalian CNS remains unclear. While oligodendrocytes have been implicated as inhibitors of CNS regeneration (Chen et al., Nature 403: 434-439, 2001; GrandPre et al., Nature 403: 439-444, 2000; Fournier et al., Nature 409: 341-346, 2001), the role of astrocytes in this process remains incompletely understood.

The data available in the literature are contradictory and give a picture of a substantial complexity. Recently, it has been suggested that in the two regions of the adult CNS in which new neurons are generated, the dentate gyrus of the hippocampus and in the subventricular zone, astrocytes positively control neurogenesis (Song et al., Nature 417: 39-44, 2002). Alternately, it has been shown that when the IF proteins of GFAP and vimentin are genetically ablated in mice, reactive astrocytes cannot form IFs (Eliasson et al., J. Biol. Chem. 274: 23996-24006, 1999) and this alters both the characteristics of the formed post-traumatic glial scar (Pekny et al., J. Cell Biol. 145: 503-514, 1999) and leads to a major improvement in the integration of transplanted cells into CNS (Kinouchi et al., Nat Neurosci. 6:863-8, 2003). The latter implicates astrocytes in the adult CNS as inhibitors of neuroregeneration.

Thus, the question of whether RG is supportive or inhibitory with respect to neurogenesis is not clear. On one hand, the presence of reactive astrocytes surrounding growing axons in the adult mammalian CNS suggests that these cells are an obstacle to regeneration (Eng and Ghirnikar, Brain Pathol. 4: 229-237, 1994). Similarly, the ablation of dividing subpopulation of reactive astrocytes in mice by genetic means leads to somewhat improved signs of regeneration (Bush et al, Neuron. 23:297-308, 1999). On the other hand, the latter situation also leads to more prominent signs of neurodegeneration (Bush et al., supra). Moreover, both mechanical and ischemic injury of the adult mammalian CNS, which triggers RG in the affected region, was found to improve both the incorporation and subsequent differentiation of the grafted cells into neurons and glia (Nishida et al., Invest. Opthalmol. Vis. Sci. 41: 4268-4274, 2000; Kurimoto et al., Neurosci. Lett. 306: 57-60, 2001). Thus, experimental data exists implying both positive and negative role of reactive gliosis in regeneration in the adult mammalian CNS.

As the foregoing discussion of literature indicates, there has been significant investigation into the biology of mammalian neurogenesis for the purposes of developing effective measures for diagnosing, treating, and preventing mammalian neurodegenerative diseases and stimulating neurogenesis. Methods of screening for modulators of the CNS and stimulating the regenerative capacity of the mammalian CNS are lacking.

### SUMMARY OF THE INVENTION

The present invention generally relates to materials and methods for identifying modulators of neurogenesis and neuroregeneration, and in particular, modulators that stimulate neurogenesis or neuroregeneration or modulate the central nervous system in a mammalian subject to create an environment more permissive or neurogenesis or neuroregeneration. The modulators themselves also are aspects of the invention, as are mixtures of the modulators; mixtures of modulators with any other neurotropic factors; and compositions comprising the modulators in pharmaceutically acceptable carriers. The invention also provides methods for using said modulators in the treatment or prevention of neurodegenerative diseases and neurological injury including modality which improves migration from the transplantation site, integration and/or survival of neural stem cells or immature neural or glial cells upon their transplantation into brain, retina, or spinal cord as a means of therapy. In a related aspect, the invention includes methods of using the modulators to improve cognitive function in any neurological disorder or following any neurological injury or slow the loss of such function due to aging or neurodegenerative disorder such as prion diseases, amyloidoses, Alzheimer's Disease, and the like.

Thus, in one aspect, the present invention provides a method of screening for a Complement 3a receptor (C3aR) agonist comprising steps of (a) contacting a composition comprising a C3aR polypeptide in the presence and absence of a test agent; (b) measuring C3aR activation in the presence and absence of the test agent; and (c) selecting as a C3aR agonist a test agent that induces C3aR activation.

In one variation, the measuring step comprises steps of: (i) measuring binding between the test agent and the C3aR polypeptide; and (ii) measuring C3aR internalization, wherein a test agent that binds C3aR and induces C3aR internalization is identified as a C3aR agonist. In another variation, the measuring step comprises steps of: (i) measuring binding between the test agent and the C3aR polypeptide; and (ii) measuring C3aR signaling, wherein a test agent that binds C3aR and induces C3aR intracellular signaling is identified as a C3aR agonist. A variety of techniques for measuring receptor signaling are described herein and/or known in the art.

In some embodiments, the contacting step comprises contacting the C3aR polypeptide with a composition comprising a C3a polypeptide, and wherein binding between the test agent and the C3aR is measured by comparing C3a binding to C3aR in the presence and absence of the test agent, wherein decreased C3a binding to C3aR in the presence of the test agent indicates binding between the test agent and C3aR.

Any composition containing the receptor may be suitable for practice of the invention. The C3aR is expressed on the surface of cells and preferred compositions include whole cells or membrane fractions of the cells, where the receptor is present in a non-denatured form in the membrane. Human and other species orthologs of this (and other receptor targets described herein) are known, and any such variation of the receptor may be employed to practice the invention. Human forms of the receptor are preferred. Moreover, it will be appreciated that modifications (insertions, deletions, substitutions) of natural mammalian receptor sequences can be made using recombinant techniques, and such modified receptors can be used to practice the invention as well.

In another embodiment, the invention includes a method of screening for a C3a receptor (C3aR) agonist, comprising steps of: (a) contacting a composition comprising a C3aR polypeptide with a composition comprising a C3a polypeptide in the presence and absence of a test agent; (b) measuring and comparing C3a binding to C3aR in the presence and absence of the test agent, wherein a test agent that inhibits C3a binding to C3aR is selected as a C3aR binding agent; (c) contacting a C3aR binding agent of step (b) to a cell that expresses C3aR on its surface; and (d) measuring and comparing receptor activation or receptor internalization in the presence and absence of the C3aR binding agent, wherein increased receptor activation or internalization in the presence of a C3aR binding agent compared to the absence identifies that C3aR binding agent as a C3aR agonist. In one aspect, the composition comprising C3aR polypeptide comprises a cell expressing the C3aR polypeptide on its surface. In another aspect, the cell is recombinantly modified to express elevated levels of C3aR on its surface. In one embodiment, the cell is a cell transformed or transfected with a polynucleotide encoding C3aR and wherein the cell expresses C3aR encoded by the polynucleotide on its surface. In another aspect, the composition comprising C3aR polypeptide comprises isolated cell membranes from cells that express C3aR polypeptide on their surfaces. In another embodiment, the contacting step generally comprises contacting a suspension of cell membranes from cells comprising C3aR with C3a and the test agent. In a further aspect, the binding between C3a and C3aR is detected by measuring a C3a-induced change to said cell. In one embodiment, the C3a-induced change in said cell is selected from the group consisting of a change in intracellular calcium ion concentration, a conversion of GTP to GDP, a change in cAMP concentration, cellular chemotaxis, and H₂O₂ production. In a further embodiment of the method, the C3aR polypeptide comprises an amino acid sequence at least 90% identical to the C3aR amino acid sequence selected from the group consisting of SEQ ID NOS: 5-7. In another embodiment, the C3a polypeptide comprises an amino acid sequence at least 90% identical to the C3a amino acid sequence selected from the group consisting of SEQ ID NOS: 2-3.

In another aspect, the present invention provides a method of screening for a complement 5a receptor (C5aR) agonist comprising the steps of (a) contacting a composition comprising a C5aR polypeptide in the presence and absence of a test agent; (b) measuring C5aR activation in the presence and absence of the test agent; and (c) selecting a test agent that induces C5aR activation. Such a measuring step generally comprises the steps of: (i) measuring binding between the test agent and the C5aR polypeptide; and (ii) measuring C5aR internalization, wherein a test agent that binds C5aR and induces C5aR internalization is identified as a C5aR agonist. In another embodiment, the measuring step comprises the steps of: (i) measuring binding between the test agent and the C5aR polypeptide; and (ii) measuring C5aR signaling, wherein a test agent that binds C5aR and induces C5aR intracellular signaling is identified as a C5aR agonist. In various embodiments of the method, the contacting step comprises contacting the C5aR polypeptide with a composition comprising a C5a polypeptide, and wherein binding between the test agent and the C5aR is measured by comparing C5a binding to C5aR in the presence and absence of the test agent, wherein decreased C5a binding to C5aR in the presence of the test agent indicates binding between the test agent and C5aR.

In another embodiment, the invention includes a method of screening for a C5a receptor (C5aR) agonist, comprising steps of: (a) contacting a composition comprising a C5aR polypeptide with a composition comprising a C5a polypeptide in the presence and absence of a test agent; (b) measuring and comparing C5a binding to C5aR in the presence and absence of the test agent, wherein a test agent that inhibits C5a binding to C5aR is selected as a C5aR binding agent; (c) contacting a C5aR binding agent of step (b) to a cell that expresses C5aR on its surface; and (d) measuring and comparing receptor activation or receptor internalization in the presence and absence of the C5aR binding agent, wherein increased receptor activation or internalization in the presence of a C5aR binding agent compared to the absence identifies that C5aR binding agent as a C5aR agonist. In one aspect, the composition comprising C5aR polypeptide comprises a cell expressing the C5aR polypeptide on its surface. In another aspect, the cell is recombinantly modified to express elevated levels of C5aR on its surface. In one embodiment, the cell is a cell transformed or transfected with a polynucleotide encoding C5aR and wherein the cell expresses C5aR encoded by the polynucleotide on its surface. In another aspect, the composition comprising C5aR polypeptide comprises isolated cell membranes from cells that express C5aR polypeptide on their surfaces. In another embodiment, the contacting step generally comprises contacting a suspension of cell membranes from cells comprising C5aR with C5a and the test agent. In a further aspect, the binding between C5a and C5aR is detected by measuring a C5a-induced change to said cell. In one embodiment, the C5a-induced change in said cell is selected from the group consisting of a change in intracellular calcium ion concentration, a conversion of GTP to GDP, a change in cAMP concentration, cellular chemotaxis, and H₂O₂ production. In a further embodiment of the method, the C5aR polypeptide comprises an amino acid sequence at least 90% identical to the C5aR amino acid sequence selected from the group consisting of SEQ ID NOS: 12-16. In another embodiment, the C5a polypeptide comprises an amino acid sequence at least 90% identical to the C5a amino acid sequence selected from the group consisting of SEQ ID NOS: 9-10.

The present invention also includes methods of screening for an inhibitor of insulin-like growth factor binding protein 4 (IGFBP-4) comprising the steps of (a) contacting a cell or cell component expressing IGFBP-4 polypeptide in the presence and absence of a test agent; (b) measuring IGFBP-4 expression or activity in the presence and absence of the test agent; and (c) selecting a test agent that reduces IGFBP-4 expression or activity. In one embodiment, the cell is recombinantly modified to express elevated levels of IGFBP-4 polynucleotide or polypeptide. In another embodiment, the cell is transformed or transfected with a polynucleotide encoding IGFBP-4 and wherein the cell expresses IGFBP-4 messenger RNA or polypeptide encoded by the polynucleotide. In a further embodiment, the IGFBP-4 polypeptide comprises an amino acid sequence at least 90% identical to the IGFBP-4 amino acid sequence selected from the group consisting of SEQ ID NOS: 18-20.

In still another variation, the invention includes a method of screening for an inhibitor of insulin-like growth factor binding protein 4 (IGFBP-4) comprising steps of: (a) contacting an IGFBP-4 polypeptide and an IGFBP-4 binding partner in the presence and absence of a test agent; (b) measuring and comparing binding between the IGFBP-4 polypeptide and the binding partner in the presence and absence of the test agent; and (c) selecting as an inhibitor a test agent that reduces IGFBP-4 binding to the binding partner. A preferred IGFBP-4 binding partner for use in practicing the invention is a naturally occurring binding partner, such as an IGF polypeptide, for which sequences are known in the art and available from public databases such as the GenBank or SwissProt databases.

In some variations, the IGFBP-4 polypeptide comprises: (1) an amino acid sequence at least 90% identical to the IGFBP-4 amino acid sequence selected from the group consisting of SEQ ID NOS: 13-15; or (2) a fragment of (1) that binds to an IGF polypeptide.

As with other screening methods described herein, preferred variations include additional steps to verify that initial lead compounds are effective in cell based or animal (including human) environments. Thus, such methods optionally include additional steps of culturing a neuron or neural stem cell or glial cell in the presence and absence of the inhibitor of IGFBP-4; measuring and comparing cell growth or survival or differentiation in the presence and absence of the inhibitor of IGFBP-4; and selecting an inhibitor of IGFBP-4 that promotes increased survival or growth or differentiation of said neuron or neural stem cell or glial cell.

Optionally, the method further comprises a step of making an IGFBP-4 inhibitor composition comprising the inhibitor of IGFBP-4 and a pharmaceutically acceptable carrier. The method may further comprise administering the IGFBP-4 inhibitor composition to a mammalian subject, and screening for neurological effects of the IGFBP-4 inhibitor on the subject. In one variation, the mammalian subject suffers from a neurological trauma, and wherein the mammalian subject is screened for neurological regeneration at a trauma site. In another variation, the mammalian subject suffers from neurological degeneration, and the mammalian subject is screened for inhibition of the degeneration.

The present invention also provides methods of screening for an inhibitor of UNC-51-Like Kinase (ULK) comprising the steps of (a) contacting a cell or cell component expressing ULK polypeptide in the presence and absence of a test agent; (b) measuring ULK expression or activity in the presence and absence of the test agent; and (c) selecting a test agent that reduces ULK expression or activity. In one embodiment, the cell is recombinantly modified to express elevated levels of ULK polynucleotide or polypeptide. In another embodiment, the cell is transformed or transfected with a polynucleotide encoding ULK and wherein the cell expresses ULK messenger RNA or polypeptide encoded by the polynucleotide. In a further embodiment, the ULK polypeptide comprises an amino acid sequence at least 90% identical to the ULK amino acid sequence selected from the group consisting of SEQ ID NOS: 22-23.

The invention also includes a method of selecting an ETBR antagonist that promotes neurogenesis or neuroregeneration comprising steps of culturing a neuron or neural stem cell or glial cell in the presence and absence of the ETBR antagonist; measuring and comparing cell growth or survival or differentiation in the presence and absence of the ETBR antagonist; and selecting an ETBR antagonist that promotes increased survival or growth or differentiation of said neuron or neural stem cell or glial cell.

In one aspect, the methods of the invention comprise steps of culturing a neuron or neural stem cell in the presence and absence of the agonists or antagonists as set out above; measuring and comparing cell growth or survival or differentiation in the presence and absence of the agonist or antagonist; and selecting an agonist or antagonist that promotes increased survival or growth or differentiation of said neuron or neural stem cell. In a certain embodiment, the cell is selected from the group consisting of a hippocampal neuron or neural stem cell, a subventricular neuron or neuron stem cell, a cortical neuron or neuron stem cell, and a neuroblastoma cell.

The invention also includes methods of making compositions comprising the agonists or antagonists and pharmaceutically acceptable carriers. Such compositions may also include the use of at least one additional factor which promotes neurogenesis or neuroregeneration selected from the group consisting of: NGF, BDNF, NT-3, 4, 5, or 6, CNTF, IGFI, IGFII, GDNF, GPA, bFGF, TGFB, and apolipoprotein E. In a further embodiment, the invention includes administering the agonist or antagonist compositions to a mammalian subject, and screening for neurological effects of the agonist or antagonist on the subject. In one embodiment, the mammalian subject suffers from a neurological trauma and is screened for neurological regeneration at a trauma site. In another embodiment, the mammalian subject suffers from neurological degeneration and is screened for inhibition of the degeneration.

In a further aspect, a mammalian subject is treated with a therapeutically effective amount of a modulator, which is an amount that is sufficient to induce a desired response in the treated subject. Thus, a biologically or therapeutically effective amount of a modulator may be the amount that interferes with physiological activity of the treated mammal in a non-lethal manner. A preferred modulator for use in the treatment methods is an agonist of C3aR, C5aR, or an antagonist of ETBR. Another preferred modulator for use in the treatment methods is an inhibitor of IGFBP-4 or ULK expression or activity. The invention is not limited to particular means for delivering the modulator to a mammal, nor is the invention limited as to the compositions comprising the modulator which may be delivered.

Modulators are identified as test compounds that alter, i.e., increase (agonist) or decrease (antagonist), a receptor function, such as a binding property of a receptor or an activity such as G protein-mediated signal transduction or membrane localization. The composition may contain an isolated receptor; alternatively, the composition may contain a receptor in association with, e.g., an intact cell or cell portion, such as a membrane. The methods of the invention embrace ligands that are attached to a label, such as a radiolabel (e.g., ¹²⁵I, ³⁵S, ³²P, ³³P, ³H), a fluorescence label, a chemiluminescence label, an enzymatic label and an immunogenic label.

In various embodiments of the methods of the invention, assays may take the form of an ion flux assay, a yeast growth assay, a non-hydrolyzable GTP assay such as a [³⁵S]-GTPγS assay, a cAMP assay, an inositol triphosphate assay, a diacylglycerol assay, a luciferase assay, a FLIPR assay for intracellular Ca2+ concentration, a mitogenesis assay, an ELISA, as well as other binding or function-based assays that are generally known in the art.

Alternatively, modulators of receptor activity may be identified as compounds that interfere with the expression of a receptor, either through inhibiting transcription of the DNA or translation of the corresponding mRNA. Expression of the receptor can be monitored by any methods known in the art, including Western blot analysis using polyclonal or monoclonal antibodies to the receptor or Northern blot analysis or quantitative polymerase chain reaction (PCR) using suitable probes or primers based on the sequence of the receptor gene. In particular, agents that interfere with the expression of gene products include anti-sense polynucleotides and ribozymes that are complementary to the gene sequences. The invention further embraces methods to modulate transcription of gene products of the invention through use of oligonucleotide-directed triplet helix formation.

An additional aspect of the invention includes any compound or composition identified according to methods of the invention as a compound that stimulates neurogenesis or neuroregeneration. To the extent any such compound is already described in the literature, the invention nonetheless includes compositions comprising the compound as well as uses of the compound or compositions for therapeutic and prophylactic purposes described herein. Preferred compositions include the compound mixed with a pharmaceutically acceptable carrier, and or mixed with any other neural growth factor or other factor identified according to the invention as beneficial for neurogenesis or neuroregeneration.

Thus, the invention includes use of a C3aR agonist or a C5aR agonist in the manufacture of a medicament for the treatment or prevention of a neurological degeneration in a mammalian subject. The invention also includes use of a C3aR agonist or a C5aR agonist in the manufacture of a medicament for the treatment of a neurological injury or trauma in a mammalian subject. Similarly, the invention includes a method of inhibiting neuronal degeneration comprising administering to a mammalian subject at risk for neuronal degeneration an agent selected from the group consisting of a C3aR agonist, a C5aR agonist, and combinations thereof. The invention similarly includes a method of improving cognitive function or slowing loss of cognitive function comprising administering to a mammalian subject an agent selected from the group consisting of a C3aR agonist, a C5aR agonist, and combinations thereof.

In related embodiments, the invention includes use of an IGFBP-4 antagonist, a ULK antagonist, or an ETBR antagonist in the manufacture of a medicament for the treatment or prevention of a neurological degeneration in a mammalian subject. The invention further includes use of an IGFBP-4 antagonist, a ULK antagonist, or an ETBR antagonist in the manufacture of a medicament for the treatment of a neurological injury or trauma in a mammalian subject. In a related embodiment, the invention provides a method of inhibiting neuronal degeneration comprising administering to a mammalian subject at risk for neuronal degeneration an agent selected from the group consisting of an IGFBP-4 antagonist, a ULK antagonist, an ETBR antagonist, and combinations thereof. Similarly, the invention includes a method of improving cognitive function or slowing loss of cognitive function comprising administering to a mammalian subject an agent selected from the group consisting of an IGFBP-4 antagonist, a ULK antagonist, an ETBR antagonist, and combinations thereof.

In addition to inhibitors that can be identified using screening methods of the invention, the invention also includes a variety of inhibitors that can be manufactured using known molecular biological techniques, based on knowledge of the protein, gene, cDNA, or mRNA structure of various targets described herein. Thus, the invention includes antagonists such as the following (alone or in combination with each other):

(a) antibody substances that bind to IGFBP-4, ULK, and ETBR;

(b) antisense oligonucleotides that hybridize to IGFBP-4, ULK, or ETBR genomic DNA or mRNA and inhibit expression of IGFBP-4, ULK, or ETBR;

(c) interfering RNA molecules that inhibit expression of IGFBP-4, ULK, or ETBR; and

(d) aptamers that inhibit expression of IGFBP-4, ULK, or ETBR.

With respect to targets for which agonists are desired, agonist antibody substances are contemplated as aspects of the invention.

All of the foregoing modulators are useful for practicing the therapeutic methods of the invention. Such methods can be practiced on any mammal, including laboratory animals, zoo animals, livestock, dogs, cats, other pets, and the like. Treatment of human subjects is specifically contemplated.

Additional features and variations of the invention will be apparent to those skilled in the art from the entirety of this application, including the detailed description and the claims as originally presented (which are incorporated into the summary of invention by reference), and all such features are intended as aspects of the invention. Moreover, features of the invention described herein can be re-combined into additional embodiments that also are intended as aspects of the invention, irrespective of whether the combination of features is specifically mentioned above as an aspect or embodiment of the invention. For the sake of brevity, various details that are applicable to multiple embodiments have not been repeated for every embodiment. Variations reflecting combinations and rearrangements of the embodiments described herein are intended as aspects of the invention. Also, only those limitations that are described herein as critical to the invention should be viewed as such; variations of the invention lacking features that have not been described herein as critical are intended as aspects of the invention.

With respect to aspects of the invention that have been described as a set or genus, every individual member of the set or genus is intended, individually, as an aspect of the invention, even if, for brevity, every individual member has not been specifically mentioned herein. When aspects of the invention that are described herein are being selected from a genus, it should be understood that the selection can include mixtures of two or more members of the genus.

In addition to the foregoing, the invention includes, as an additional aspect, all embodiments of the invention narrower in scope in any way than the variations specifically described herein. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### DETAILED DESCRIPTION OF THE PREFERRED EMBODIMENTS

The present invention includes materials and methods for screening for agents to regulate or manipulate neuroregeneration and neurogenesis. The present invention also involves use of these agents in the treatment of neurodegenerative diseases and neurological injury. DNA array technology was used to compare the gene expression profiles of wild-type mice (standard, regeneration-inhibiting) and GFAP −/− Vimentin −/− mice (regeneration-supporting model) to identify candidate genes to target to modulate in neurogenesis and neuroregeneration in the adult CNS. These molecular targets are useful to derive agents to promote neurogenesis and neuroregeneration. The same targets also may be useful to screen for agents to inhibit growth of CNS tumors.

Definitions

The term “agent that induces C3aR activation” refers to any molecule or molecules (e.g., nucleic acid, peptide, polypeptide, binding agent, antibody, peptibody, small molecule, etc.) which can act directly or indirectly to upregulate expression or signaling of the C3aR polypeptide. The term also encompasses agonists and other modulators which induce, stimulate, or enhance C3aR expression or signaling in cells that express C3aR.

The term “agent that induces C5aR activation” refers to any molecule or molecules (e.g., nucleic acid, peptide, polypeptide, binding agent, antibody, peptibody, small molecule, etc.) which can act directly or indirectly to upregulate expression or signaling of the C5aR polypeptide. The term also encompasses agonists and other modulators which induce, stimulate, or enhance C5aR expression or signaling in cells that express C5aR.

The term “agent that reduces IGFBP-4 expression or activity” refers to any molecule or molecules (e.g., nucleic acid, peptide, polypeptide, binding agent, antibody, peptibody, small molecule, etc.) which can act directly or indirectly to downregulate or inhibit expression or activity of the IGFBP-4 polypeptide. The term also encompasses antagonists and other modulators which reduce, inhibit, or decrease IGFBP-4 expression or activity.

The term “agent that reduces ULK expression or activity” refers to any molecule or molecules (e.g., nucleic acid, peptide, polypeptide, binding agent, antibody, peptibody, small molecule, etc.) which can act directly or indirectly to downregulate or inhibit expression or activity of the ULK polypeptide. The term also encompasses antagonists and other modulators which reduce, inhibit, or decrease ULK expression or activity.

The term “agent that reduces ETBR activation” refers to any molecule or molecules (e.g., nucleic acid, peptide, polypeptide, binding agent, antibody, peptibody, small molecule, etc.) which can act directly or indirectly to downregulate or inhibit expression or signaling of the ETBR polypeptide. The term also encompasses antagonists and other modulators which reduce, inhibit, or decrease ETBR expression or signaling in cells that express ETBR.

The term “nucleic acid construct” refers to any nucleic acid molecule or molecules such as an isolated cDNA or genomic DNA protein encoding polynucleotide alone or in conjunction with a vector, promoter, enhancer, terminator, etc. This term includes, but is not limited to, DNA, RNA, oligonucleotides, including upstream and downstream regulators of nucleic acid expression.

The term “therapeutically effective amount” in the context of neurological diseases or conditions described herein refers to an amount effective to achieve measurable improvement (compared to an untreated control) as assessed by any relevant medical parameter used to evaluate subjects receiving treatment for the disease or condition.

The term “expression vector” refers to a vector which (when transformed or transfected into a suitable host cell) contains nucleic acid sequences which direct and/or control the expression, transcription, and/or translation of heterologous nucleic acid sequences that are inserted into the vector.

The term “host cell” is used to refer to a cell which has been transformed or transfected, or is capable of being transformed or transfected, with a nucleic acid. The term also includes the transgenic progeny of the transformed or transfected cell, whether or not the progeny is identical in morphology or in genetic make-up to the original parent.

The terms “pharmaceutically acceptable carrier” as used herein refer to one or more formulation materials suitable for accomplishing or enhancing the delivery of a substance (as a pharmaceutical composition) to a mammalian (preferably a human) subject.

The term “selective binding agent” refers to a molecule or molecules having binding specificity for a selected target. As used herein the term “specific binding” refers to the ability to bind to a target such as a polypeptide in vivo and no significantly cross-react with other molecules (e.g. other polypeptides in vivo). Selective binding agents may also bind orthologs (species homologs) of the specifically identified polypeptides. Lack of cross-reactivity is preferably measured under physiological conditions unless otherwise stated and can be demonstrated using conventional assays (e.g. Western blots) or by measuring dissociation constants.

The term “transduction” is used to refer to the transfer of genes from one bacterium to another, usually by a phage. “Transduction” also refers to the acquisition and transfer of eukaryotic cellular sequences by retroviruses.

The term “transfection” is used to refer to the uptake of foreign or exogenous DNA by a cell, and a cell has been “transfected” when the exogenous DNA has been introduced inside the cell membrane. A number of transfection techniques are well known in the art and are disclosed herein. See, for example, Graham et al., Virology, 52: 456, 1973; Sambrook et al., Molecular Cloning, A Laboratory Manual, Cold Spring Harbor Laboratories (New York, 1989); Davis et al., Basic Methods in Molecular Biology, Elsevier, 1986; and Chu et al., Gene, 13: 197, 1981. Such techniques can be used to introduce one or more exogenous DNA moieties into suitable host cells.

The term “transformation” as used herein refers to a change in a cell's genetic characteristics, and a cell has been transformed when it has been modified to contain new DNA. For example, a cell is transformed where it is genetically modified from its native state. Following transfection or transduction, the transforming DNA may recombine with that of the cell by physically integrating into a chromosome of the cell, it may be maintained transiently as an episomal element without being replicated, or it may replicate independently as a plasmid. A cell is considered to have been stably transformed when the DNA is replicated with the division of the cell.

The term “vector” is used to refer to any molecule (e.g., nucleic acid, plasmid, or virus) used to transfer coding information to a host cell.

The term “central nervous system” or “CNS” includes the brain, spinal cord, and retina.

The term “neurological regeneration” includes CNS regeneration or neuroregeneration and refers to one of several types of events that lead to functional improvement of the CNS. CNS improvement can manifest itself as an improvement, stabilization, or slowed-down deterioration of functions such as cognition, vision, etc. The cellular basis for neuroregeneration can be e.g. increased proliferation (or slower death) of neural stem cells; generation of new neurons and/or glial cells (referred to as neurogenesis and gliogenesis, respectively); formation of new, or stabilization of the existing, neuronal synapses (synaptic regeneration); or re-growth of severed axons (axonal regeneration).

The term “neurological trauma” includes CNS trauma and refers to one of several types of physical or pathological events including ischemic and hypoxic damage, toxic damage, and damage connected with a metabolic impairment (e.g. diabetes and diabetic retinopathy).

The term “neurological degeneration” includes CNS degeneration and refers to one of several types of neurodegenerative diseases and disorders including e.g. Alzheimer's disease, Parkinson's disease, multiple sclerosis, Jacob-Creutzfelt's disease and other prion diseases), epilepsy, and aging.

### A. C3aR AND C5aR AS TARGETS FOR MODULATING NEUROGENESIS AND NEUROREGENERATION

The present invention includes a method of screening for complement 3a receptor (C3aR) or complement 5a receptor (C5aR) modulators to identify molecules useful to modulate neurogenesis and neuroregeneration and for using said modulators in the treatment of neurodegenerative diseases and neurological injury including modality, which improves migration from the transplantation site, integration and/or survival of neural stem cells, or immature neural or glial cells, upon their transplantation into the CNS as a means of therapy.

Complement, a component of the humoral immune system, is involved in inflammation, opsonization, and cytolysis. More than 20 plasma proteins participate in the activation and regulation of complement, most of them functioning as enzymes, enzyme inhibitors, or enzyme cofactors. In addition, there are more than 10 membrane proteins that regulate complement activation or serve as receptors for proteolytic fragments generated during activation of the cascade. The complement cascade can be activated via the classical, lectin, or alternative pathway, resulting in the formation of C3-convertase, an enzymatic complex that activates the central molecule of the cascade, the third component, C3. The proteolytic activation of C3 generates C3a, a small fragment with anaphylatoxic properties, and C3b, a larger fragment that binds to an activating surface and triggers the terminal part of the cascade, culminating in the assembly of the terminal complement complex on the target surface and generating the most powerful complement-derived anapyhlatoxin, C5a.

The complement system has recently been proposed to participate in tissue regeneration in several different systems. Hippocampal and cortical neurons have also been shown to express receptors for complement fragments C3a and C5a (Davoust et al., Glia 26:201-211, 1999; O'Barr et al., J. Immunol. 166:4154-4162, 2001. Recently, the inventors used immunohistochemistry and polyclonal antibodies against C3aR and C5aR on neural stem cells in vitro and in vivo (sections of mouse brain containing the main neurogenic zone (subventricular of lateral ventricles), and in a focal brain ischemia model in mice deficient in C3 (C3−/−) Pekna et al., Scan. J. Immunol. 47:25-29, 1998), to find that C3−/− mice had 30-50% fewer neural progenitor cells and 25% fewer newly formed neurons in the prenumbra than controls, and larger infarct volumes. C3−/− mice also had 24% fewer migrating neural progenitor cells in the subventricular zone (the main source of progenitor cells in the adult brain). These findings suggested that the complement system promotes neuroregeneration after cerebral ischemia. Therefore, the present invention contemplates the use of C3aR and C5aR agonists to stimulate neurogenesis or neuroregeneration and provides methods of screening for C3aR and C5aR agonists.

Exemplary nucleotide and amino acid sequences for C3a, C5a, C3aR, and C5aR are set forth in the sequence listing and in Table I as summarized below:

TABLE 1
C3A AND C3AR SEQUENCES

| IDENTITY | SPECIES | SEQ ID NOS: |
|---|---|---|
| C3a | Homo sapiens (mRNA) | 1 |
| | NM_00064 | |
| C3a | Homo sapiens | 2 |
| | NP_000055 | |
| C3a | Rattus norvegicus | 3 |
| | P01026 | |
| C3aR | Homo sapiens (DNA) | 4 |
| | AY268431 | |
| C3aR | Homo sapiens Q16581 | 5 |
| C3aR | Cavia porcellus O88680 | 6 |
| C3aR | Mus musculus O09047 | 7 |
| C5a | Homo sapiens (mRNA) | 8 |
| | NM_001735 | |
| C5a | Homo sapiens P01031 | 9 |
| C5a | Mus musculusP06684 | 10 |
| C5aR | Homo sapiens (DNA) | 11 |
| | X58674 | |
| C5aR | Homo sapiens P21730 | 12 |
| C5aR | Gorilla gorilla | 13 |
| | CAA66317 | |
| C5aR | Cavia porcellus O70129 | 14 |
| C5aR | Rattus norvegicus | 15 |
| | P97520 | |
| C5aR | Mus musculus P30993 | 16 |

### B. IGFBP-4 AS A TARGET FOR MODULATING NEUROGENESIS AND NEUROREGENERATION

IGFBP-4 belongs to a family of six secreted IGF binding proteins. They all have similar domain organizations but differ in their ability to adhere to cellular surfaces and to their effect in the IGF regulating system. IGF-I and IGF-II are two growth promoting peptides that have been shown to improve survival and growth of various cell types including neurons (Zhou et al., Endocrinol. 178:177-193, 2003). IGF-I and IGF-II exert their functions through binding to the cellular receptors IGF-RII and IGF-RII, which are two trans-membrane proteins with tyrosine kinase activity (Zhou et al., 2003, supra). IGF-I and IGF-II in the extracellular environment are transported by IGF binding proteins. The affinity between IGFs and IGF binding protein is always equal to or greater than IGF's affinity for its cellular receptors. The affinity is decreased and thereby regulated through phosphorylation, glycosylation, proteolysis or adherence to cell surface or extracellular matrix. This leads to local increase in IGF bioavailability (Zhou et al., 2003, supra).

IGFBP-4 is the smallest of the IGF binding proteins. IGFBP-4 exists in biological fluid as a doublet with a molecular weight of 24 kDa in its non-glycosylated form and 28 kDa in its glycosylated form (Zhou et al., 2003, supra). IGFBP-4 binds equally to IGF-I and IGF-II and it has solely shown inhibitory effects on the IGF system. IGFBP-4 binding to IGF makes IGFBP-4 a substrate for the protease PAPP-A. Upon a single site cleavage with PAPP-A, IGF is released to the local environment. Because PAPP-A reversibly adheres to several different cell types, IGFBP-4's effect could be locally directed (Zhou et al., 2003, supra). The genetic expression of IGFBP-4 is thought to be developmentally regulated and its expression has been shown to be regulated by hormones and cytokines in a tissue specific manner (Zhou et al., 2003, supra).

In the hippocampus of the uninjured control mouse brain, astrocytes were found to be highly positive for IGFBP-4. Likewise, in the hippocampus of the injured control mouse brain (after entorhinal cortex lesions), reactive astrocytes were found to be highly positive for IGFBP-4. However, both non-reactive and reactive astrocytes in GFAP−/−vim−/− (knockout) mice, that exhibit a regeneration-permissive environment, are negative for IGFBP-4. Therefore, the present invention contemplates the use of IGFBP-4 antagonists to stimulate neurogenesis or neuroregeneration and provides methods of screening for IGFBP-4 antagonists.

Exemplary nucleotide and amino acid sequences for IGFBP-4 are set forth in the sequence listing and in Table 2 as summarized below:

TABLE 2
IGFBP-4 SEQUENCES

| IDENTITY | SPECIES | SEQ ID NOS: |
|---|---|---|
| IGFBP-4 | Homo sapiens (mRNA) | 17 |
| | NM_001552 | |
| IGFBP-4 | Homo sapiens | 18 |
| | NP_001543 | |
| IGFBP-4 | Rattus norvegicus | 19 |
| | P21744 | |
| IGFBP-4 | Mus musculus P47879 | 20 |

### C. UNC-51-LIKE KINASE (ULK) AS A TARGET FOR MODULATING NEUROGENESIS AND NEUROREGENERATION

ULK1 has been shown to be involved in axonal elongation (Tomoda, Neuron 24: 833-846, 1999), which may involve the interaction of ULK1 and GABA-A receptor associated protein (Okazaki, Brain Res Mol Brain Res. 85: 1-12, 2000). The present invention contemplates the use of ULK antagonists to stimulate neurogenesis or neuroregeneration and provides methods of screening for ULK antagonists.

Exemplary nucleotide and amino acid sequences for ULK are set forth in the sequence listing and in Table 3 as summarized below:

TABLE 3
ULK SEQUENCES

| IDENTITY | SPECIES | SEQ ID NOS: |
|---|---|---|
| ULK | Homo sapiens (mRNA) | 21 |
| | NM_003565 | |
| ULK | Homo sapiens | 22 |
| | NP_003556 | |
| ULK | Mus musculus | 23 |
| | AAH57121 | |

### D. ENDOTHELIN RECEPTOR B (ETBR) AS A TARGET FOR MODULATING NEUROGENESIS AND NEUROREGENERATION

Endothelin receptor B (ETBR) belongs to a family of receptors that bind the endothelin peptides I, II, and III. ETBR is normally highly expressed by reactive astrocytes and weakly by endothelial cells (Ishikawa et al, Eur. J. Neurosci. 9:895-901, 1997; Baba, Life Sci. 62:1711-1715, 1998; Koyama et al., Glia 26:268-271, 1999; Peters et al., Exp. Neurol., 180:1-13, 2003). Recently, it was shown that administration of endothelin 1, an ETBR agonist, leads to astrocyte hypertrophy, while Bosenthan, an ETBR antagonist, reduced astrocyte hypertrophy in CNS trauma (Rogers et al., Glia 41:180-190, 2003). It was also suggested that the blockage of ETBR may attenuate the formation of an astrocytic glial scar (Rogers et al., supra). Several ETBR antagonists, with diversified specificity for the ETB and ETA receptors are known, such as those described in patent publications DE19858779, WO9911629, WO9857938, WO9711942, U.S. Pat. No. 5,866,568, and EP0562599, all incorporated herein by reference in their entireties.

Expression of ETBR was examined in GFAP−/−Vim−/− knockout mice (Wilhelmsson et al., J. Neuroscience (in press), 2004). Perfused brains were postfixed for one day at 4° C. in paraformaldehyde and horizontal vibratome sections (50 μm) were made from the hippocampus, and stored in a cryoprotectant (50% 0.05M sodium phosphate, pH 7.3, 30% ethylene glycol, 20% glycerol) at −20° C. After several washes in PBS, the sections were incubated in 0.05% glycine in PBS for 1 h at room temperature and permeabilized overnight in PBS containing 0.5% Tween 20 and 1% BSA at 4° C. Rabbit anti-ETBR antibody (Alomone Labs Ltd, Jerusalem, Israel), and mouse anti-GFAP antibody (clone GA5, Sigma-Aldrich), both diluted 1:100, followed by Alexa 488-conjugated anti-rabbit and Alexa 568-conjugated anti-mouse antibodies (Molecular Probes), both diluted 1:500, were used for immunohistochemical staining. For nuclear staining, propidium iodide (Sigma-Aldrich) or TO PRO-3 (Molecular Probes) was added to the last wash before mounting.

In GFAP−/−Vim−/− knockout mice (regeneration-permissive environment), ETBR protein was undetectable on reactive astrocytes, while very abundant on reactive astrocytes of normal mice (Wilhelmsson et al., 2004, supra). However, ETBR was present on endothelial cells in both wild-type and knockout mice. Interestingly, it has been found that ETBR co-localizes with intermediate filaments, a very surprising finding for a cell surface receptor (Wilhelmsson et al., 2004, supra). These results therefore suggest that the upregulation or activation of ETBR in reactive astrocytes requires an intermediate filament network, and in the absence of ETBR upreguLlation or activation, there is improved post-traumatic regeneration (Wilhelmsson et al., 2004, supra). Therefore, the present invention contemplates the use of ETBR antagonists to stimulate neurogenesis or neuroregeneration, and provides methods for identifying such antagonists.

Exemplary nucleotide and amino acid sequences for ETBR are set forth in the sequence listing and in Table 4 as summarized below:

TABLE 4
ETBR SEQUENCES

| IDENTITY | SPECIES | SEQ ID NOS: |
|---|---|---|
| ETBR | Homo sapiens (DNA) | 24 |
| | CA772539 | |
| ETBR | Homo sapiens P24530 | 25 |
| ETBR | Bos taurus P28088 | 26 |
| ETBR | Rattus norvegicus | 27 |
| | P21451 | |
| ETBR | Mus musculus P48302 | 28 |

### E. ASSAYING FOR MODULATORS OF RECEPTOR BINDING AND ACTIVATION

The present invention has several aspects, one of which is modulating the activity of C3a receptor (C3aR), C5a receptor (C5aR) and endothelin receptor B (ETBR). C3aR and C5aR are receptors for the chemotactic and anaphylactic peptides, C3a and C5a, which are mediators of complement inflammatory functions. ETBR belongs to a family of receptors that bind the endothelin peptides I, II, and III.

Assays contemplated by the invention include both binding assays and activity assays; these assays may be performed in conventional or high throughput formats. Modulator screens are designed to identify stimulatory and inhibitory agents. The sources for potential agents to be screened include natural sources, such as a cell extract (e.g., invertebrate cells including, but not limited to, bacterial, fungal, algal, and plant cells) and synthetic sources, such as chemical compound libraries or biological libraries such as antibody substance or peptide libraries. Agents are screened for the ability to either stimulate or inhibit the activity. Binding assays are used to detect receptor binding activity to ligands. Both functional and binding assays of receptor activity are readily adapted to screens for modulators such as agonist (stimulatory) and antagonist (inhibitory) compounds.

The invention contemplates a multitude of assays to screen and identify modulators, such as agonists and antagonists, of ligand binding to receptors. In one example, the receptor is immobilized and interaction with a binding partner is assessed in the presence and absence of a candidate modulator. In another example, the binding partner is immobilized and the receptor is solubilized. In yet another example, interaction between the receptor and its binding partner is assessed in a solution assay, both in the presence and absence of a candidate modulator. An antagonist is identified as a compound that decreases binding between the receptor and its binding partner and/or decreases receptor signaling, while an agonist is identified as a compound that increases binding between the receptor and its binding partner and/or promotes receptor signaling. Another contemplated assay involves a variation of the di-hybrid assay wherein a modulator of protein/protein interactions is identified by detection of a positive signal in a transformed or transfected host cell.

Candidate modulators for screening according to contemplated by the invention include any chemical compounds, including libraries of chemical compounds. There are a number of different libraries used for the identification of small molecule modulators, including: (1) chemical libraries, (2) natural product libraries, and (3) combinatorial libraries comprised of random peptides, oligonucleotides or organic molecules. Chemical libraries consist of random chemical structures, or analogs of known compounds, or analogs of compounds that have been identified as “hits” or “leads” in prior drug discovery screens, some of which may be derived from natural products or from non-directed synthetic organic chemistry. Natural product libraries are collections of microorganisms, animals, plants, or marine organisms which are used to create mixtures for screening by: (1) fermentation and extraction of broths from soil, plant or marine microorganisms or (2) extraction of plants or marine organisms. Natural product libraries include polyketides, non-ribosomal peptides, and variants (non-naturally occurring) thereof. For a review, see Science 282:63-68 (1998). Combinatorial libraries are composed of large numbers of peptides, oligonucleotides, or organic compounds as a mixture. These libraries are relatively easy to prepare by traditional automated synthesis methods, PCR, cloning, or synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of modulators through use of the various libraries described herein permits modification of the candidate “hit” (or “lead”) to optimize the capacity of the “hit” to modulate activity.

Candidate modulators contemplated by the invention can be designed and include soluble forms of binding partners, as well as chimeric, or fusion, proteins thereof. A “binding partner” as used herein broadly encompasses non-peptide modulators, peptide modulators (e.g., neuropeptide variants), antibodies (including monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR and/or antigen-binding sequences, which specifically recognize a polypeptide of the invention), antibody fragments, and modified compounds comprising antibody domains that are immunospecific for the expression product of the identified GPCR-like gene.

A number of assays are known in the art that can identify chemical compounds that bind to or interact with a receptor. Such assays are useful, for example, in methods of identifying candidate modulators described herein, or in methods for identifying specific ligands of a receptor. Assays that measure binding or interaction of compounds with target proteins include assays that identify compounds that inhibit unfolding or denaturation of a target protein, assays that separate compounds that bind to target proteins through affinity ultrafiltration followed by ion spray mass spectroscopy/HPLC methods or other physical and analytical methods, capillary electrophoresis assays and two-hybrid assays.

One such screening method to identify direct binding of test ligands to a target protein is described in U.S. Pat. No. 5,585,277, incorporated herein by reference. This method relies on the principle that proteins generally exist as a mixture of folded and unfolded states, and continually alternate between the two states. When a test ligand binds to the folded form of a target protein (i.e., when the test ligand is a ligand of the target protein), the target protein molecule bound by the ligand remains in its folded state. Thus, the folded target protein is present to a greater extent in the presence of a test ligand which binds the target protein, than in the absence of a ligand. Binding of the ligand to the target protein can be determined by any method which distinguishes between the folded and unfolded states of the target protein. The function of the target protein need not be known in order for this assay to be performed. Virtually any agent can be assessed by this method as a test ligand, including, but not limited to, metals, polypeptides, proteins, lipids, polysaccharides, polynucleotides and small organic molecules.

Another method for identifying ligands of a target protein is described in Wieboldt et al., Anal. Chem., 69:1683-1691 (1997), incorporated herein by reference. This technique screens combinatorial libraries of 20-30 agents at a time in solution phase for binding to the target protein. Agents that bind to the target protein are separated from other library components by simple membrane washing. The specifically selected molecules that are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Alternatively, such binding interactions are evaluated indirectly using the yeast two-hybrid system described in Fields et al., Nature, 340:245-246 (1989), and Fields et al., Trends in Genetics, 10:286-292 (1994), both of which are incorporated herein by reference. The two-hybrid system is a genetic assay for detecting interactions between two proteins or polypeptides. It can be used to identify proteins that bind to a known protein of interest, or to delineate domains or residues critical for an interaction. Variations on this methodology have been developed to clone genes that encode DNA binding proteins, to identify peptides that bind to a protein, and to screen for drugs. The two-hybrid system exploits the ability of a pair of interacting proteins to bring a transcription activation domain into close proximity with a DNA binding domain that binds to an upstream activation sequence (UAS) of a reporter gene, and is generally performed in yeast. The assay requires the construction of two hybrid genes encoding (1) a DNA-binding domain that is fused to a first protein and (2) an activation domain fused to a second protein. The DNA-binding domain targets the first hybrid protein to the UAS of the reporter gene; however, because most proteins lack an activation domain, this DNA-binding hybrid protein does not activate transcription of the reporter gene. The second hybrid protein, which contains the activation domain, cannot by itself activate expression of the reporter gene because it does not bind the UAS. However, when both hybrid proteins are present, the noncovalent interaction of the first and second proteins tethers the activation domain to the UAS, activating transcription of the reporter gene.

When the function of the receptor is unknown and no ligands are known to bind the gene product, the yeast two-hybrid assay can be used to identify proteins that bind to the receptor. In an assay to identify proteins that bind to a receptor, or fragment thereof, a fusion polynucleotide encoding both a receptor or fragment (i.e., a first protein) and a UAS binding domain may be used. In addition, a large number of hybrid genes each encoding a different second protein fused to an activation domain are produced and screened in the assay. Typically, the second protein is encoded by one or more members of a total cDNA or genomic DNA fusion library, with each second protein coding region being fused to the activation domain. This system is applicable to a wide variety of proteins, and it is not even necessary to know the identity or function of the second binding protein. The system is highly sensitive and can detect interactions not revealed by other methods; even transient interactions may trigger transcription to produce a stable mRNA that can be repeatedly translated to yield the reporter protein.

In addition, when the receptor or fragment thereof is known to interact with another protein or nucleic acid, the two-hybrid assay can be used to detect agents that interfere with the binding interaction. Expression of the reporter gene is monitored as different test agents are added to the system. The presence of an inhibitory agent, for example, results in lack of or reduction in a reporter signal.

1. Antibodies to Receptors as Modulators of Binding

Standard techniques are employed to generate polyclonal or monoclonal antibodies to receptors, and to generate useful antigen-binding fragments thereof or variants thereof. Such protocols can be found, for example, in Sambrook et al., Molecular Cloning: a Laboratory Manual. Second Edition, Cold Spring Harbor, N.Y.: Cold Spring Harbor Laboratory (1989); Harlow et al. (Eds), Antibodies A Laboratory Manual; Cold Spring Harbor Laboratory; Cold Spring Harbor, N.Y. (1988). In one embodiment, recombinant polypeptides (or cells or cell membranes containing such polypeptides) are used as antigens to generate the antibodies. In another embodiment, one or more peptides having amino acid sequences corresponding to an immunogenic portion of a receptor are used as antigen. Peptides corresponding to extracellular portions of receptors, especially hydrophilic extracellular portions, are preferred. The antigen may be mixed with an adjuvant or linked to a hapten to increase antibody production. Polyclonal and monoclonal antibodies, chimeric (e.g., humanized) antibodies, fragments of antibodies, and all other forms of antibody molecules disclosed herein are referred to collectively as antibody products.

a. Polyclonal or Monoclonal Antibodies

As one exemplary protocol, a recombinant polypeptide or a synthetic fragment thereof is used to immunize a mouse for generation of monoclonal antibodies (or larger mammal, such as a rabbit, for polyclonal antibodies). To increase antigenicity, peptides are conjugated to Keyhole Lympet Hemocyanin (Pierce), according to the manufacturer's recommendations. For an initial injection, the antigen is emulsified with Freund's Complete Adjuvant and injected subcutaneously. At intervals of two to three weeks, additional aliquots of receptor antigen are emulsified with Freund's Incomplete Adjuvant and injected subcutaneously. Prior to the final booster injection, a serum sample is taken from the immunized mice and assayed by Western blot to confirm the presence of antibodies that immunoreact with a polypeptide. Serum from the immunized animals may be used as a polyclonal antisera or used to isolate polyclonal antibodies that recognize a receptor. Alternatively, the mice are sacrificed and their spleens are removed for generation of monoclonal antibodies.

To generate monoclonal antibodies, the spleens are placed in 10 ml serum-free RPMI 1640, and single-cell suspensions are formed by grinding the spleens in serum-free RPMI 1640, supplemented with 2 mM L-glutamine, 1 mM sodium pyruvate, 100 Units/ml penicillin, and 100 μg/ml streptomycin (RPMI) (Gibco, Canada). The cell suspensions are filtered and washed by centrifugation and resuspended in serum-free RPMI. Thymocytes taken from three naive Balb/c mice are prepared in a similar manner and used as a Feeder Layer. NS-1 myeloma cells, kept in log phase in RPMI with 10% (FBS(Hyclone Laboratories, Inc., Logan, Utah) for three days prior to fusion, are centrifuged and washed as well.

To produce hybridoma fusions, spleen cells from the immunized mice are combined with NS-1 cells and centrifuged, and the supernatant is aspirated. The cell pellet is dislodged by tapping the tube, and 2 ml of 37° C. PEG 1500 (50% in 75 mM HEPES, pH 8.0) (Boehringer-Mannheim) is stirred into the pellet, followed by the addition of serum-free RPMI. Thereafter, the cells are centrifuged and resuspended in RPMI containing 15% FBS, 100 μM sodium hypoxanthine, 0.4 μM aminopterin, 16 μM thymidine (HAT) (Gibco), 25 Units/ml IL-6 (Boehringer-Mannheim) and 1.5×106 thymocytes/ml and plated into 10 Corning flat-bottom 96-well tissue culture plates (Corning, Corning N.Y.).

On days 2, 4, and 6 after the fusion, 100 μl of medium is removed from the wells of the fusion plates and replaced with fresh medium. On day 8, the fusions are screened by ELISA, testing for the presence of mouse IgG that binds to a receptor polypeptide. Selected fusions are further cloned by dilution until monoclonal cultures producing anti-receptor antibodies are obtained.

b. Receptor-Neutralizing Antibodies from Phage Display

Receptor-neutralizing antibodies are generated by phage display techniques such as those described in Aujame et al., Human Antibodies, 8(4):155-168 (1997); Hoogenboom, TIBTECH, 15:62-70 (1997); and Rader et al., Curr. Opin. Biotechnol., 8:503-508 (1997), all of which are incorporated by reference. For example, antibody variable regions in the form of Fab fragments or linked single chain Fv fragments are fused to the amino terminus of filamentous phage minor coat protein pIII. Expression of the fusion protein and incorporation thereof into the mature phage coat results in phage particles that present an antibody on their surface and contain the genetic material encoding the antibody. A phage library comprising such constructs is expressed in bacteria, and the library is screened for target-specific phage-antibodies using a labeled or immobilized target peptide or polypeptide as antigen-probe.

c. Receptor-Neutralizing Antibodies from Transgenic Animals

Receptor-neutralizing antibodies are generated in transgenic animals, such as mice, essentially as described in Bruggemann et al., Immunol. Today 17(8):391-97 (1996) and Bruggemann et al., Curr. Opin. Biotechnol. 8:455-58 (1997). Transgenic mice carrying V-gene segments in germline configuration, and expressing the transgenes in their lymphoid tissue, are immunized with a polypeptide composition using conventional immunization protocols. Hybridomas are generated from B cells of the immunized mice using conventional protocols and screened to identify hybridomas secreting anti-receptor antibodies (e.g., as described above).

2. Assays to Identify Modulators of Receptor Activity

A number of approaches exist for the discovery of novel compounds that are binding partners for receptors. Each of the general approaches is compatible with high throughput screening (i.e., HTS) formats, which are preferred formats for identifying ligands and other binding partners of receptor polypeptides, such as modulators of receptor activity. The first approach involves measuring the binding of a known ligand, preferably labeled with a radiolabel, to a preparation that contains the receptor, either found in native tissue or based on expression of the gene encoding the receptor, typically in a heterologous system. The recombinant system involves the expression of a recombinant receptor. Another approach to the identification of receptor binding partners involves the measurement of the activity of the receptor, which may be influenced by either the binding of ligands that are agonists, and elevate receptor activity, or by the binding of antagonists, which interfere with agonist binding, thereby reducing the level of receptor activity. As for binding assays, recombinant systems are the presently preferred forms for function-based assays.

a. Receptor Binding Assays (RBA) as High Throughput Screening (HTS) Systems for Drug Discovery

The literature is replete with examples of the use of radiolabeled ligands in HTS binding assays for drug discovery (see Williams, Med. Res. Rev. 11:147-184 (1991); Sweetnam et al., J. Nat. Prod. 56:441-455 (1993) for review). It is also possible to screen for novel neuroregeneration compounds with radiolabeled ligands in HTS binding screens (Geary et al., 1999). Other reasons that recombinant receptors are preferred for HTS binding assays include better specificity (higher relative purity) and ability to generate large amounts of receptor material (see Hodgson, Bio/Technology 10:973-980 (1992)).

A variety of heterologous systems are available for expression of recombinant receptors and are well known to those skilled in the art. Such systems include bacteria (Strosberg et al., Trends in Pharm. Sci. 13:95-98 (1992)), yeast (Pausch, Trends in Biotech. 15:487-494 (1997)), several kinds of insect cells (Vanden Broeck, Intl. Rev. Cytol. 164:189-268 (1996)), amphibian cells (Jayawickreme et al., Curr. Opin. Biotechnol. 8:629-634 (1997)) and several mammalian cell lines (CHO, HEK293, COS, etc.; see Gerhardt et al., Eur. J. Pharmacol. 334:1-23 (1997); Wilson et al., Brit. J. Pharmacol. 125:1387-1392 (1998)). These examples do not preclude the use of other possible cell expression systems, including cell lines obtained from nematodes (WO 98/37177).

A receptor expressed in one of the described recombinant systems can be used for HTS binding assays in conjunction with its defined ligand. The identified peptide is labeled with a suitable radioisotope, including, but not limited to, ¹²⁵I (preferred; see Geary et al., 1999), ³H, ³⁵S or ³²P, by methods that are well known to those skilled in the art. Alternatively, the peptides may be labeled by well-known methods with a suitable fluorescent label (Baindur et al., Drug Dev. Res. 33:373-398 (1994); Rogers, Drug Disc. Today 2:156-160 (1997)). Radioactive ligand specifically bound to the receptor in membrane preparations made from cells expressing the recombinant protein can be detected in HTS assays in one of several standard ways, including filtration of the receptor-ligand complex to separate bound ligand from unbound ligand (Williams, 1991; Sweetnam et al., 1993). Alternative methods include a scintillation proximity assay (SPA) or a FlashPlate format in which such separation is unnecessary (Nakayama et al., Drug Disc. & Dev. 1:85-91 (1998); Boss et al., J. Biomol. Screening 3:285-292 (1998)). Binding of fluorescent ligands can be detected in various ways, including fluorescence energy transfer (FRET), direct spectrophotofluorometric analysis of bound ligand, or fluorescence polarization (see Rogers, 1997; Hill, Curr. Opin. Drug Disc. & Dev. 1:92-97 (1998)).

b. Response-Based Receptor HTS systems

Activation of heterologous G-protein-coupled receptors expressed in recombinant systems results in a variety of biological responses, which are typically mediated by G proteins expressed in the host cells. Agonist binding to a GPCR results in exchange of bound GDP for GTP at a binding site on the G subunit; one can use a radioactive, non-hydrolyzable derivative of GTP, such as [³⁵S]GTPγS, to measure binding of an agonist to the receptor. (Seifert et al., Eur. J. Biochem. 255:369-382 (1998).) One can also use this binding to measure the ability of antagonists to bind to the receptor by decreasing binding of GTP [³⁵] in the presence of a known agonist.

The G proteins required for functional expression of heterologous GPCRs can be native constituents of the host cell or can be introduced through well-known recombinant technology. The G proteins can be intact or chimeric. Often, a nearly universally competent G protein (e.g., Gα16) is used to couple any given receptor to a detectable response pathway. G protein activation results in the stimulation or inhibition of other native proteins, events that can be linked to a measurable response.

Examples of such biological responses include, but are not limited to, the following responses: the ability to survive in the absence of a limiting nutrient in specifically engineered yeast cells (Pausch, 1997); changes in intracellular Ca2+ concentration as measured by fluorescent dyes (Murphy et al., Curr. Opin. Drug Disc. & Dev. 1: 192-199 (1998)). Fluorescence changes can also be used to monitor ligand-induced changes in membrane potential or intracellular pH; an automated system suitable for HTS has been described for these purposes (Schroeder et al., J. Biomol. Screening 1:75-80 (1996)). Melanophores prepared from Xenopus laevis show a ligand-dependent change in pigment organization in response to heterologous GPCR activation; this response is adaptable to HTS formats (Jayawickreme et al., 1997). Assays are also available for the measurement of common second messengers, including cAMP, phosphoinositides and arachidonic acid. Set forth in the following subsections are exemplary function-based assays for identifying modulators (agonists and antagonists) of GPCR-like receptor activity. Among the modulators that can be identified by these assays are natural ligand compounds of the receptor; synthetic analogs and derivatives of natural ligands; antibodies, antibody fragments, and/or antibody-like compounds derived from natural antibodies or from antibody-like combinatorial libraries; and/or synthetic compounds identified by high throughput screening of libraries; and other libraries known in the art. All modulators that bind GPCR-like receptors are useful for identifying GPCR-like polypeptides in tissue samples (e.g., for diagnostic purposes, pathological purposes, and other purposes known in the art). Agonist and antagonist modulators are useful for up-regulating and down-regulating GPCR-like receptor activity, respectively, for purposes described herein. GPCR-like receptor binding partners also may be used to deliver a therapeutic compound or a label to cells that express a GPCR-like receptor (e.g., by attaching the compound or label to the binding partner). The assays may be performed using single putative modulators; they may also be performed using a known agonist in combination with candidate antagonists (or visa versa).

c. cAMP Assays

In one type of assay, levels of cyclic adenosine monophosphate (cAMP) are measured in GPCR-like receptor-transfected cells that have been exposed to candidate modulator compounds. Protocols for cAMP assays have been described in the literature. [See, e.g., Sutherland et al., Circulation, 37:279 (1968); Frandsen, et al., Life Sciences, 18:529-541 (1976); Dooley et al., J. Pharm. & Exp. Therap., 283(2):735-41 (1997); and George et al., J. Biomol. Screening, 2(4):235-40 (1997).] An exemplary protocol for such an assay, using an Adenylyl Cyclase Activation FlashPlate® Assay from NEN™ Life Science Products, is set forth below.

Briefly, the GPCR-like receptor coding sequence (e.g., a cDNA or intronless genomic DNA) is subcloned into a commercial expression vector, such as pzeoSV2 (Invitrogen, San Diego, Calif.), and transiently transfected into Chinese Hamster Ovary (CHO) cells using known methods, such as the transfection reagent FuGENE 6 (Boehringer-Mannheim) and the transfection protocol provided in the product insert. The transfected CHO cells are seeded into the 96-well microplates of the FlashPlate® assay kit, which are coated with solid scintillant to which antisera to cAMP has been bound. For a control, some wells are seeded with wild type (untransfected) CHO cells. Other wells on the plate receive various amounts of cAMP standard solution for use in creating a standard curve.

One or more test compounds are added to the cells in each well, with water and/or compound-free medium/diluent serving as a control. To screen for antagonists, the test compound may be added to the wells prior to adding a known agonist/ligand, or the test compound may be premixed with a known agonist before adding the mixture to the wells. After treatment, cAMP is allowed to accumulate in the cells for exactly 15 minutes at room temperature. The assay is terminated by the addition of lysis buffer containing [¹²⁵]-labeled cAMP, and the plate is counted using a Packard Topcount™ 96-well microplate scintillation counter. Unlabeled cAMP from the lysed cells (or from standards) competes with the fixed amounts of [¹²⁵I]-cAMP for antibody bound to the plate. A standard curve is constructed, and cAMP values for the unknowns are obtained by interpolation. Changes in intracellular cAMP level of the cells in response to exposure to a test compound are indicative of GPCR receptor modulating activity. Modulators that act as agonists at receptors which couple to the Gs subtype of G proteins will stimulate production of cAMP, leading to a measurable 3-10 fold increase. Agonists of receptors which couple to the Gi/o subtype of G proteins will inhibit forskolin-stimulated cAMP production, leading to a measurable decrease of 50-100%. Modulators that act as antagonists will reverse these effects at receptors that are either constitutively active or activated by known agonists.

d. Luciferase Reporter Gene Assay

The photoprotein luciferase provides another useful tool for assaying for modulators of GPCR-like receptor activity. Cells (e.g., CHO cells or COS 7 cells) are transiently co-transfected with both a GPCR-like receptor expression construct (e.g., a GPCR-like receptor in pzeoSV2 (Invitrogen, San Diego, Calif.)) and a reporter construct which includes a luciferase coding region downstream from a transcription factor, either the cAMP-response element (CRE), AP-1, or NF-κB. Agonist binding to receptors coupled to the Gs subtype of G proteins leads to increases in cAMP, activating the CRE transcription factor and resulting in expression of the luciferase gene. Agonist binding to receptors coupled to the Gq subtype of G proteins leads to production of diacylglycerol that activates protein kinase C, which activates the AP-1 or NF-κB transcription factors resulting in expression of the luciferase gene. Expression levels of luciferase reflect the activation status of the signaling events. [See generally George et al., Journal of Biomolecular Screening, 2(4): 235-40 (1997); and Stratowa et al., Curr. Opin. Biotechnology, 6: 574-81 (1995).] Luciferase activity may be quantitatively measured using, e.g., luciferase assay reagents that are commercially available from Promega (Madison, Wis.).

In one exemplary assay, CHO cells are plated in 24-well culture dishes at a density of 100,000 cells/well one day prior to transfection and cultured at 37° C. in αMEM (Gibco/BRL, Gaithersburg, Md.) supplemented with 10% fetal bovine serum, 2 mM glutamine, 10 Units/ml penicillin and 10 μg/ml streptomycin. Cells are transiently co-transfected with both a GPCR receptor expression construct and a reporter construct containing the luciferase gene. The reporter plasmids CRE-luciferase, AP-1-luciferase and NF-κB-luciferase may be purchased from Stratagene (LaJolla, Calif.). Transfections are performed using FuGENE 6 transfection reagent (Boehringer-Mannheim), following the protocol provided in the product insert. Cells transfected with the reporter construct alone are used as a control. Twenty-four hours after transfection, cells are washed once with phosphate buffered saline (PBS) pre-warmed to 37° C. Serum-free αMEM is then added to the cells either alone (control) or with one or more candidate modulators and the cells are incubated at 37° C. for five hours. To screen for antagonists, the candidate modulator may be added to the cell cultures prior to adding a known agonist/ligand or the candidate modulator may be pre-mixed with a known agonist before adding the mixture to the cells. Thereafter, cells are washed once with ice-cold PBS and lysed by the addition of 100 μl of lysis buffer/well (from the luciferase assay kit, Promega, Madison, Wis.). After incubation for 15 minutes at room temperature, 15 μl of the lysate is mixed with 50 μl substrate solution (Promega) in an opaque white 96-well plate, and the luminescence is read immediately on a Wallace model 1450 MicroBeta scintillation and luminescence counter (Wallace Instruments, Gaithersburg, Md.).

Differences in luminescence in the presence versus the absence of a candidate modulator compound are indicative of modulating activity. Receptors that are either constitutively active or activated by agonists typically give a 3-20 fold stimulation of luminescence compared to cells transfected with the reporter gene alone. Modulators that act as antagonists will reverse these effects at receptors that are either constitutively active or activated by known agonists.

e. Intracellular Calcium Measurement using FLIPR

Changes in intracellular calcium levels are another recognized indicator of G protein-coupled receptor activity, and such assays can be employed to assay for modulators of GPCR-like receptor activity. For example, CHO cells stably transfected with a GPCR-like receptor expression construct are plated at a density of 4×104 cells/well in Packard black-walled 96-well plates specially designed to isolate fluorescent signals to individual wells. The cells are incubated for 60 minutes at 37° C. in modified Dulbecco's PBS (D-PBS), containing 36 mg/L pyruvate and 1 g/L glucose with the addition of 1% fetal bovine serum and one of four calcium indicator dyes (Fluo-3™ AM, Fluo-4™ AM, Calcium Green™-1AM, or Oregon Green™ 488 BAPTA-1 AM) at a concentration of 4 μM. Plates are washed once with modified D-PBS without 1% fetal bovine serum and incubated for 10 minutes at 37° C. to remove residual dye from the cellular membrane. In addition, a series of washes with modified D-PBS without 1% fetal bovine serum is performed immediately prior to activation of the calcium response.

Calcium response is initiated by the addition of one or more candidate receptor agonist compounds, or a positive control such as a calcium ionophore A23187 (10 μM), or ATP (4 μM). Fluorescence is measured by Molecular Device's FLIPR with an argon laser, excitation at 488 nm. [See, e.g., Kuntzweiler et al., Drug Development Research, 44(1): 14-20 (1998).] To screen for antagonists, a test compound or agent is added to the cell prior to adding a known agonist/ligand, or the test compound may be pre-mixed with a known agonist before adding the mixture to the cells. The F-stop for the detector camera may be set at 2.5 and the length of exposure is about 0.4 msec. Basal fluorescence of cells is measured for 20 seconds prior to addition of agonist, ATP, or A23187, and is subtracted from the response signal. The calcium signal is measured for approximately 200 seconds, taking readings every two seconds. The calcium ionophore and ATP increase the calcium signal 200% above baseline levels. In general, activated GPCRs increase the calcium signal approximately 10-15% above baseline signal. Antagonists inhibit or eliminate such activation.

f. Mitogenesis Assay

In mitogenesis assays, the ability of candidate modulators to induce or inhibit GPCR-like receptor-mediated cell growth is determined. [See, e.g., Lajiness et al., J. Pharmacol. Exper. Ther. 267(3): 1573-81 (1993).] For example, CHO cells stably expressing a GPCR-like receptor are seeded into 96-well plates at a density of 5000 cells/well and grown at 37° C. in αMEM with 10% fetal calf serum for 48 hours, at which time the cells are rinsed twice with serum-free αMEM. After rinsing, 80 μl of fresh αMEM, or αMEM containing a known mitogen, is added along with 20 μl αMEM containing varying concentrations of one or more test compounds diluted in serum-free medium. As controls, some wells on each plate receive serum-free medium alone, and some receive medium containing 10% fetal bovine serum. Untransfected cells or cells transfected with the vector alone also may serve as controls.

After culture for 16-18 hours, 1 μCi/well of [3H]-thymidine is added to the wells and cells are incubated for an additional 2 hours at 37° C. The cells are trypsinized and harvested onto filter mats with a cell harvester (Tomtec) and the filters are counted in a Betaplate counter. The incorporation of [³H]-thymidine in serum-free test wells is compared to the results achieved in cells stimulated with serum. Use of multiple concentrations of test compounds permits creation and analysis of dose-response curves using the non-linear, least squares fit equation: A=B×[C/(D+C)]+G where A is the percent of serum stimulation; B is the maximal effect minus baseline; C is the EC₅₀; D is the concentration of the compound; and G is the maximal effect. Parameters B, C and G are determined by Simplex optimization.

Agonists that bind to the receptor are expected to increase [3H]-thymidine incorporation into cells, showing up to 80% of the response to serum. Antagonists that bind to the receptor will inhibit the stimulation seen with a known agonist by up to 100%.

g. [³⁵S]GTPγS Binding Assay

Because G protein-coupled receptors signal through intracellular G proteins whose activity involves GTP/GDP binding and hydrolysis, measurement of binding of the non-hydrolyzable GTP analog [³⁵S]GTPγS in the presence and absence of putative modulators provides another indicator of modulator activity. [See, e.g., Kowal, et al., Neuropharmacology, 37: 179-87 (1998).]

In one exemplary assay, cells stably transfected with a GPCR-like receptor expression construct are grown in 10 cm dishes to subconfluence, rinsed once with 5 ml of ice-cold Ca2+/Mg2+-free PBS, and scraped into 5 ml of the same buffer. Cells are pelleted by centrifugation (500×g, 5 minutes), resuspended in 25 mM Tris, pH 7.5, 5 mM EDTA, 5 mM EDTA, pH 7.5 (TEE), and frozen in liquid nitrogen. After thawing, the cells are homogenized using a Dounce homogenizer (one ml TEE per plate of cells), and centrifuged at 1,000×g for 5 minutes to remove nuclei and unbroken cells.

The homogenate supernatant is centrifuged at 20,000×g for 20 minutes to isolate the membrane fraction, and the membrane pellet is washed once with TEE and resuspended in binding buffer (20 mM HEPES, pH 7.5, 150 mM NaCl, 10 mM MgCl2, 1 mM EDTA). The resuspended membranes can be frozen in liquid nitrogen and stored at −70° C. until use.

Aliquots of cell membranes prepared as described above and stored at −70° C. are thawed, homogenized, and diluted into buffer containing 20 mM HEPES, 10 mM MgCl2, 1 mM EDTA, 120 mM NaCl, 10 μM GDP, and 0.2 mM ascorbate at a concentration of 10-50 μg/ml. In a final volume of 90 μl, homogenates are incubated with varying concentrations of putative modulator compounds or 100 μM GTP for 30 minutes at 30° C. and then placed on ice. To each sample, 10 μl guanosine 5′-O-(3[³⁵S]thio) triphosphate (NEN, 1200 Ci/mmol; [³⁵S]-GTPγS), was added to a final concentration of 100-200 pM. Samples are incubated at 30° C. for an additional 30 minutes, and then the reaction is stopped by the addition of 1 ml of 10 mM HEPES, 10 mM MgCl₂, pH 7.4, 4° C.

Samples are filtered over Whatman GF/B filters and filters are washed with 20 ml ice-cold 10 mM HEPES, pH 7.4, 10 mM MgCl2. Filters are counted by liquid scintillation spectroscopy. Nonspecific binding of [³⁵S]-GTPγS is measured in the presence of 100 μM GTP and subtracted from the total. Compounds are selected that modulate the amount of [³⁵S]-GTPγS binding in the cells, compared to untransfected control cells. Activation of receptors by agonists gives up to a five-fold increase in [³⁵S]GTPγS binding. This response is blocked by antagonists.

### F. ASSAYING FOR OTHER MODULATORS OF POLYPEPTIDE ACTIVITY

Particular aspects of the present invention contemplate the modulation of IGFBP-4 and ULK polypeptide expression or activity. In particular, methods of identifying modulators of IGFBP-4 and ULK polypeptide expression or activity are provided in further detail herein below.

In some situations, it may be desirable to identify molecules that are modulators, i.e., agonists or antagonists, of the expression or activity of IGFBP-4 and ULK polypeptides. Natural or synthetic molecules that modulate IGFBP-4 and ULK polypeptides may be identified using one or more screening assays, such as those described herein. Such molecules may be administered either in an ex vivo manner, or in an in vivo manner by injection, or by oral delivery, implantation device, or the like. “Test molecule(s)” refers to the molecule(s) that is/are under evaluation for the ability to modulate (i.e., increase or decrease) the activity of an IGFBP-4 and ULK polypeptide. Most commonly, a test molecule will interact directly with an IGFBP-4 and ULK polypeptide. However, it is also contemplated that a test molecule may also modulate IGFBP-4 and ULK polypeptide activity indirectly, such as by affecting IGFBP-4 and ULK gene expression, or by binding to an IGFBP-4 and ULK binding partner (e.g., receptor, co-factor, or ligand). In one embodiment, a test molecule will bind to an IGFBP-4 and ULK polypeptide with an affinity constant of at least about 10⁻⁶ M, preferably about 10⁻⁸ M, more preferably about 10⁻⁹ M, and even more preferably about 10¹⁰ M.

Methods for identifying compounds which interact with an IGFBP-4 and ULK polypeptide are encompassed by the present invention. In certain embodiments, an IGFBP-4 and ULK polypeptide is incubated with a test molecule under conditions which permit the interaction of the test molecule with an IGFBP-4 and ULK polypeptide, and the extent of the interaction can be measured. The test molecule(s) can be screened in a substantially purified form or in a crude mixture.

In certain embodiments, an IGFBP-4 or ULK antagonist may be a protein, peptide, carbohydrate, lipid, or small molecular weight molecule which interacts with an IGFBP-4 or ULK polypeptide or an IGFBP-4 or ULK polynucleotide to regulate its expression or activity. Molecules which regulate IGFBP-4 or ULK polypeptide or IGFBP-4 or ULK polynucleotide expression or activity include nucleic acids which are complementary to nucleic acid encoding an IGFBP-4 and ULK polypeptide, or are complementary to nucleic acids sequences which direct or control the expression or activity of an IGFBP-4 and ULK polypeptide, and which act as antisense regulators of expression or activity.

Once a set of test molecules has been identified as interacting with an IGFBP-4 or ULK polypeptide or an IGFBP-4 or ULK polynucleotide, the molecules may be further evaluated for their ability to increase or decrease IGFBP-4 and ULK polypeptide activity. The measurement of the interaction of test molecules with an IGFBP-4 or ULK polypeptide or an IGFBP-4 or ULK polynucleotide may be carried out in several formats, including cell-based binding assays, membrane binding assays, solution-phase assays and immunoassays. In general, test molecules are incubated with an IGFBP-4 or ULK polypeptide or an IGFBP-4 or ULKpolynucleotide for a specified period of time, and IGFBP-4 or ULK polypeptide activity is determined by one or more assays for measuring biological activity.

The interaction of test molecules with IGFBP-4 and ULK polypeptides may also be assayed directly using polyclonal or monoclonal antibodies in an immunoassay. Alternatively, modified forms of IGFBP-4 and ULK polypeptides containing epitope tags as described herein may be used in immunoassays.

In the event that IGFBP-4 and ULK polypeptides display biological activity through an interaction with a binding partner (e.g., IGF, a receptor, a ligand or a co-factor), a variety of in vitro assays may be used to measure the binding of an IGFBP-4 or ULK polypeptide to the corresponding binding partner (such as a selective binding agent, receptor, ligand, or co-factor). These assays may be used to screen test molecules for their ability to increase or decrease the rate and/or the extent of binding of an IGFBP-4 or ULK polypeptide to its binding partner. In one assay, an IGFBP-4 or ULK polypeptide is immobilized in the wells of a microtiter plate. Radiolabeled an IGFBP-4 or ULK binding partner (for example, iodinated IGFBP-4 or ULK binding partner) and the test molecule(s) can then be added either one at a time (in either order) or simultaneously to the wells. After incubation, the wells can be washed and counted using a scintillation counter, for radioactivity to determine the extent to which the binding partner bound to an IGFBP-4 or ULK polypeptide. Typically, the molecules will be tested over a range of concentrations, and a series of control wells lacking one or more elements of the test assays can be used for accuracy in the evaluation of the results. An alternative to this method involves reversing the “positions” of the proteins, i.e., immobilizing an IGFBP-4 or ULK binding partner to the microtiter plate wells, incubating with the test molecule and radiolabeled IGFBP-4 or ULK polypeptide, and determining the extent of IGFBP-4 or ULK polypeptide binding. See, for example, Chapter 18, Current Protocols in Molecular Biology, Ausubel et al., eds., John Wiley & Sons, New York, N.Y. (1995).

As an alternative to radiolabelling, an IGFBP-4 or ULK polypeptide or its binding partner may be conjugated to biotin and the presence of biotinylated protein can then be detected using streptavidin linked to an enzyme, such as horseradish peroxidase (HRP) or alkaline phosphatase (AP), that can be detected colorimetrically, or by fluorescent tagging of streptavidin. An antibody directed to an IGFBP-4 or ULK polypeptide or to an IGFBP-4 or ULK binding partner and conjugated to biotin may also be used and can be detected after incubation with enzyme-linked streptavidin linked to AP or HRP.

An IGFBP-4 or ULK polypeptide or an IGFBP-4 or ULK-like binding partner can also be immobilized by attachment to agarose beads, acrylic beads or other types of such inert solid phase substrates. The substrate-protein complex can be placed in a solution containing the complementary protein and the test compound. After incubation, the beads can be precipitated by centrifugation, and the amount of binding between an IGFBP-4 or ULK polypeptide and its binding partner can be assessed using the methods described herein. Alternatively, the substrate-protein complex can be immobilized in a column, and the test molecule and complementary protein are passed through the column. The formation of a complex between an IGFBP-4 and ULK polypeptide and its binding partner can then be assessed using any of the techniques set forth herein, i.e., radiolabelling, antibody binding or the like.

Another in vitro assay that is useful for identifying a test molecule which increases or decreases the formation of a complex between an IGFBP-4 or ULK polypeptide and an IGFBP-4 or ULK binding partner is a surface plasmon resonance detector system such as the BIAcore assay system (Pharmacia, Piscataway, N.J.). The BIAcore system may be carried out using the manufacturer's protocol. This assay essentially involves the covalent binding of either (1) an IGFBP-4 or ULK polypeptide or (2) an IGFBP-4 or ULK binding partner to a dextran-coated sensor chip which is located in a detector. The test compound and the other complementary protein can then be injected, either simultaneously or sequentially, into the chamber containing the sensor chip. The amount of complementary protein that binds can be assessed based on the change in molecular mass which is physically associated with the dextran-coated side of the sensor chip; the change in molecular mass can be measured by the detector system.

In some cases, it may be desirable to evaluate two or more test compounds together for their ability to increase or decrease the formation of a complex between a an IGFBP-4 or ULK polypeptide and an IGFBP-4 or ULK binding partner. In these cases, the assays set forth herein can be readily modified by adding such additional test compound(s) either simultaneous with, or subsequent to, the first test compound. The remainder of the steps in the assay are set forth herein.

In vitro assays such as those described herein may be used advantageously to screen large numbers of compounds for effects on complex formation by an IGFBP-4 or ULK polypeptide and an IGFBP-4 or ULK binding partner. The assays may be automated to screen compounds generated in phage display, synthetic peptide, and chemical synthesis libraries.

Compounds which increase or decrease the formation of a complex between an IGFBP-4 or ULK polypeptide and an IGFBP-4 or ULK binding partner may also be screened in cell culture using cells and cell lines expressing either an IGFBP-4 or ULK polypeptide or an IGFBP-4 or ULK binding partner. Cells and cell lines may be obtained from any mammal, but preferably will be from human or other primate, canine, or rodent sources. The binding of an IGFBP-4 or ULK polypeptide to cells expressing an IGFBP-4 or ULK binding partner at the surface is evaluated in the presence or absence of test molecules, and the extent of binding may be determined by, for example, flow cytometry using a biotinylated antibody to an IGFBP-4 or ULK binding partner. Cell culture assays can be used advantageously to further evaluate compounds that score positive in protein binding assays described herein.

Cell cultures can also be used to screen the impact of a drug candidate. For example, drug candidates may decrease or increase the expression or activity of an IGFBP-4 or ULK gene. In certain embodiments, the amount of an IGFBP-4 or ULK polypeptide that is produced may be measured after exposure of the cell culture to the drug candidate. In certain embodiments, one may detect the actual impact of the drug candidate on the cell culture. For example, the overexpression of a particular gene may have a particular impact on the cell culture. In such cases, one may test a drug candidate's ability to increase or decrease the expression of the gene or its ability to prevent or inhibit a particular impact on the cell culture. In other examples, the production of a particular metabolic product such as a fragment of a polypeptide, may result in, or be associated with, a disease or pathological condition. In such cases, one may test a drug candidate's ability to decrease the production of such a metabolic product in a cell culture.

A yeast two hybrid system (Chien et al., Proc. Natl. Acad. Sci. USA, 88:9578-9583, 1991) can be used to identify novel polypeptides that bind to, or interact with, IGFBP-4 or ULK polypeptides. As an example, a yeast-two hybrid bait construct can be generated in a vector (such as the pAS2-1 from Clontech) which encodes a yeast GAL4-DNA binding domain fused to the IGFBP-4 and ULK polynucleotide. This bait construct may be used to screen human cDNA libraries wherein the cDNA library sequences are fused to GAL4 activation domains. Positive interactions will result in the activation of a reporter gene such as β-Gal. Positive clones emerging from the screening may be characterized further to identify interacting proteins.

### G. SELECTIVE BINDING AGENTS

The present invention also provides selective binding agents of polypeptides identified by the methods of the invention for the treatment of a pathological condition that would benefit from creating a regeneration-permissive environment to facilitate or stimulate neurogenesis or neuroregeneration. The screening methods described herein identify suitable selective binding agents useful for therapeutic purposes and for formulation and modification by pharmaceutical chemists to improve serum half-life, reduce toxicity, and increase potency. Suitable selective binding agents include, but are not limited to, antibodies and derivatives thereof, polypeptides, and small molecules. Suitable selective binding agents may be prepared using organic chemistry, biochemistry, molecular biology, and recombinant techniques. An exemplary selective binding agent of the present invention is capable of binding a certain portion of an identified polypeptide thereby increasing the expression and/or signaling of the C3aR, C5aR; inhibiting the expression or activity of IGFBP-4 or ULK; and decreasing the expression and/or signaling of ETBR.

Selective binding agents such as antibodies and antibody fragments that bind polypeptides which increase the expression and/or signaling of the C3aR, C5aR; inhibit the expression or activity of IGFBP-4 or ULK; and decrease the expression and/or signaling of ETBR are within the scope of the present invention. The antibodies may be polyclonal including monospecific polyclonal, monoclonal (MAbs), recombinant, chimeric, humanized such as CDR-grafted, human, single chain, and/or bispecific, as well as fragments, variants or derivatives thereof. Antibody fragments include those portions of the antibody which bind to an epitope on the polypeptide which increases the expression and/or signaling of the C3aR, C5aR; inhibits the expression or activity of IGFBP-4 or ULK; and decreases the expression and/or signaling of ETBR. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Polyclonal antibodies directed toward a polypeptide which increases the expression and/or signaling of the C3aR, C5aR; inhibits the expression or activity of IGFBP-4 or ULK; and decreases the expression and/or signaling of ETBR generally are produced in animals (e.g., rabbits or mice) by means of multiple subcutaneous or intraperitoneal injections of a polypeptide and an adjuvant. It may be useful to conjugate a polypeptide which increases the expression and/or signaling of the C3aR, C5aR; inhibits the expression or activity of IGFBP-4 or ULK; and decreases the expression and/or signaling of ETBR to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for antibody titer.

Monoclonal antibodies directed toward polypeptides which increase the expression and/or signaling of the C3aR, C5aR; inhibit the expression or activity of IGFBP-4 or ULK; and decrease the expression and/or signaling of ETBR are within the scope of the present invention. These antibodies are produced using any method which provides for the production of antibody molecules by continuous cell lines in culture. Examples of suitable methods for preparing monoclonal antibodies include the hybridoma methods of Kohler et al. (Nature, 256: 495-497, 1975) and the human B-cell hybridoma method (Kozbor et al., J. Immunol., 133: 3001-3005, 1984; Brodeur et al., Monoclonal Antibody Production Techniques and Applications, pp. 51-63, Marcel Dekker, Inc., New York, 1987). Also provided by the invention are hybridoma cell lines which produce monoclonal antibodies reactive with polypeptides which increase the expression and/or signaling of the C3aR, C5aR; inhibit the expression or activity of IGFBP-4 or ULK; and decrease the expression and/or signaling of ETBR.

Antibodies of the invention are screened in assays described herein for the desired agonist (C3aR or C5aR) or antagonist (IGFBP-4, ULK, and ETBR) activity. To facilitate screening, and also for diagnostic applications, in certain embodiments, antibodies which increase the expression and/or signaling of the C3aR or C5aR; inhibit the expression or activity of IGFBP-4 or ULK; and decrease the expression and/or signaling of ETBR typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, b-galactosidase, or horseradish peroxidase (Bayer et al., Meth. Enz., 184: 138-163, 1990).

Competitive binding assays rely on the ability of a labeled standard (e.g., a polypeptide, or an immunologically reactive portion thereof) to compete with the test sample analyte for binding with a limited amount of antibody. The amount of a polypeptide in the test sample is inversely proportional to the amount of standard that becomes bound to the antibodies. To facilitate determining the amount of standard that becomes bound, the antibodies typically are insolubilized before or after the competition, so that the standard and analyte that are bound to the antibodies may conveniently be separated from the standard and analyte which remain unbound.

Sandwich assays typically involve the use of two antibodies, each capable of binding to a different immunogenic portion, or epitope, of the protein to be detected and/or quantitated. In a sandwich assay, the test sample analyte is typically bound by a first antibody which is immobilized on a solid support, and thereafter a second antibody binds to the analyte, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assays). For example, one type of sandwich assay is an enzyme-linked immunosorbent assay (ELISA), in which case the detectable moiety is an enzyme.

Selective binding agents of the invention, including antibodies which act as agonists (increase the expression and/or signaling) of C3aR or C5aR, or antagonists (inhibit the expression or activity (or signaling)) of IGFBP-4 or ULK or ETBR, may be used as therapeutics. These therapeutic agents are generally agonists of a C3aR or a C5aR, or antagonists of IGFBP-4, ULK, or ETBR polypeptide.

In one embodiment, antagonist antibodies of the invention are antibodies or binding fragments thereof which are capable of specifically binding to an IGFBP-4, ULK, or ETBR polypeptide and which are capable of inhibiting or eliminating the functional activity of an IGFBP-4, ULK, or ETBR polypeptide in vivo or in vitro. In preferred embodiments, the selective binding agent, e.g., an antagonist antibody will inhibit the functional activity of an IGFBP-4, ULK, or ETBR polypeptide by at least about 50%, and preferably by at least about 80%. In another embodiment, the selective binging agent may be an antibody that is capable of interacting with an IGFBP-4, ULK, or ETBR binding partner (a ligand, co-factor, or receptor) thereby inhibiting or eliminating IGFBP-4, ULK, or ETBR activity in vitro or in vivo. Selective binding agents, including antagonist anti-IGFBP-4, -ULK, or -ETBR antibodies are identified by screening assays which are well known in the art.

### H. OTHER ANTI-LIGAND AND ANTI-RECEPTOR COMPOUNDS

The present invention also provides anti-ligand and anti-receptor compounds such as aptamer, antisense, and interference RNA techniques and therapies for the treatment of a pathological condition resulting from decreased expression or signaling of C3aR, C5aR; upregulated expression of IGFBP-4 or ULK; or increased expression or signaling of ETBR.

1. Aptamers

Recent advances in the field of combinatorial sciences have identified short polymer sequences with high affinity and specificity to a given target. For example, SELEX technology has been used to identify DNA and RNA aptamers with binding properties that rival mammalian antibodies, the field of immunology has generated and isolated antibodies or antibody fragments which bind to a myriad of compounds and phage display has been utilized to discover new peptide sequences with very favorable binding properties. Based on the success of these molecular evolution techniques, it is certain that molecules can be created which bind to any target molecule. A loop structure is often involved with providing the desired binding attributes as in the case of: aptamers which often utilize hairpin loops created from short regions without complimentary base pairing, naturally derived antibodies that utilize combinatorial arrangement of looped hyper-variable regions and new phage display libraries utilizing cyclic peptides that have shown improved results when compared to linear peptide phage display results. Thus, sufficient evidence has been generated to suggest that high affinity ligands can be created and identified by combinatorial molecular evolution techniques. For the present invention, molecular evolution techniques can be used to isolate binding constructs specific for ligands described herein. For more on aptamers, See generally, Gold, L., Singer, B., He, Y. Y., Brody. E., “Aptamers As Therapeutic And Diagnostic Agents,” J. Biotechnol. 74:5-13 (2000). Relevant techniques for generating aptamers may be found in U.S. Pat. No. 6,699,843, which is incorporated by reference in its entirety.

In some embodiments, the aptamer may be generated by preparing a library of nucleic acids; contacting the library of nucleic acids with a growth factor, wherein nucleic acids having greater binding affinity for the growth factor (relative to other library nucleic acids) are selected and amplified to yield a mixture of nucleic acids enriched for nucleic acids with relatively higher affinity and specificity for binding to the growth factor. The processes may be repeated, and the selected nucleic acids mutated and rescreened, whereby a growth factor aptamer is be identified. Nucleic acids may be screened to select for molecules that bind to more than growth factor.

2. Antisense Molecules

Another class of inhibitors that may be used in conjunction with the present invention is isolated antisense nucleic acid molecules that can hybridize to, or are complementary to, the nucleic acid molecule, nucleotide sequence, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific embodiments, antisense nucleic acid molecules are provided that comprise a sequence complementary to at least about 10, 25, 50, 100, 250 or 500 nucleotides or an entire receptor or ligand coding strand, or to only a portion thereof. Nucleic acid molecules encoding fragments, homologs, derivatives and analogs of receptor or ligand or antisense nucleic acids complementary to a receptor or ligand nucleic acid sequence are additionally provided.

In one embodiment, an antisense nucleic acid molecule is antisense to a “coding region” of the coding strand of a nucleotide sequence encoding a receptor or ligand protein (or fragments or fragment combination thereof). The term “coding region” refers to the region of the nucleotide sequence comprising codons that are translated into amino acid residues. In another embodiment, the antisense nucleic acid molecule is antisense to a “conceding region” of the coding strand of a nucleotide sequence encoding the receptor or ligand protein. The term “conceding region” refers to 5′ and 3′ sequences that flank the coding region and that are not translated into amino acids (i.e., also referred to as 5′ and 3′ untranslated regions).

Given the coding strand sequences encoding the receptor or ligand protein disclosed herein, antisense nucleic acids of the invention can be designed according to the rules of Watson and Crick or Hoogsteen base pairing. The antisense nucleic acid molecule can be complementary to the entire coding region of a ligand or receptor mRNA, but more preferably is an oligonucleotide that is antisense to only a portion of the coding or noncoding region of receptor or ligand mRNA. For example, the antisense oligonucleotide can be complementary to the region surrounding the translation start site of receptor or ligand mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following section).

The antisense nucleic acid molecules of the invention are typically administered to a subject or generated in situ such that they hybridize with or bind to cellular mRNA and/or genomic DNA encoding a receptor or ligand to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual alpha-units, the strands run parallel to each other. See, e.g., Gaultier, et al., Nucl. Acids Res., 15:6625-6641 (1987). The antisense nucleic acid molecule can also comprise a 2′-o-methylribonucleotide (see, e.g., Inoue, et al. Nucl. Acids Res., 15:6131-6148 (1987)) or a chimeric RNA-DNA analogue (see, e.g., Inoue, et al., FEBS Lett., 215:327-330 (1987)).

Production and delivery of antisense molecules are facilitated by providing a vector comprising an anti-sense nucleotide sequence complementary to at least a part of the Receptor or ligand DNA sequence. According to a yet further aspect of the invention such a vector comprising an anti-sense sequence may be used to inhibit, or at least mitigate, Receptor or ligand expression. The use of a vector of this type to inhibit Receptor or ligand expression is favored in instances where Receptor or ligand expression is associated with a particular disease state.

3. RNA Interference

Use of RNA Interference to inactivate or modulate receptor or ligand expression is also contemplated by this invention. RNA interference is described in U.S. Patent Appl. No. 2002-0162126, and Hannon, G., J. Nature, 11:418:244-51 (2002). “RNA interference,” “post-transcriptional gene silencing,” “quelling”—these terms have all been used to describe similar effects that result from the overexpression or misexpression of transgenes, or from the deliberate introduction of double-stranded RNA into cells (reviewed in Fire, A., Trends Genet 15:358-363 (1999); Sharp, P. A., Genes Dev., 13:139-141 (1999); Hunter, C., Curr. Biol., 9:R440-R442 (1999); Baulcombe, D. C., Curr. Biol. 9:R599-R601 (1999); Vaucheret, et al. Plant J. 16:651-659 (1998), all incorporated by reference. RNA interference, commonly referred to as RNAi, offers a way of specifically and potently inactivating a cloned gene.

### I. GENE THERAPY

Some of the therapeutic agents identified herein or identified by methods herein are nucleic acids or proteins that can be delivered by gene therapy (e.g. delivery of a nucleic acid or a nucleic acid encoding a therapeutic polypeptide. Additional embodiments of the present invention relate to cells and methods (e.g., homologous recombination and/or other recombinant production methods) for both the in vitro production of therapeutic polypeptides and for the production and delivery of therapeutic polypeptides by gene therapy or cell therapy. Homologous and other recombination methods may be used to modify a cell that contains a normally transcriptionally silent gene, or an under expressed gene, and thereby produce a cell which expresses therapeutically efficacious amounts of polypeptides identified by the methods of the invention.

Homologous recombination is a technique originally developed for targeting genes to induce or correct mutations in transcriptionally active genes (Kucherlapati, Prog. Nucleic Acid Res. Mol. Biol. 36:301-310, 1989). The basic technique was developed as a method for introducing specific mutations into specific regions of the mammalian genome (Thomas et al., Cell 44:419-428, 1986; Thomas et al., Cell 51:503-512, 1987; Doetschman et al., Proc. Natl. Acad. Sci. USA, 85:8583-8587, 1988) or to correct specific mutations within defective genes (Doetschman et al., Nature 330:576-578, 1987). Exemplary homologous recombination techniques are described in U.S. Pat. No. 5,272,071 (EP 9193051, EP Publication No. 505500; PCT/US90/07642, International Publication No. WO 91/09955).

Through homologous recombination, the DNA sequence to be inserted into the genome can be directed to a specific region of the gene of interest by attaching it to targeting DNA. The targeting DNA is a nucleotide sequence that is complementary (homologous) to a region of the genomic DNA. Small pieces of targeting DNA that are complementary to a specific region of the genome are put in contact with the parental strand during the DNA replication process. It is a general property of DNA that has been inserted into a cell to hybridize, and therefore, recombine with other pieces of endogenous DNA through shared homologous regions. If this complementary strand is attached to an oligonucleotide that contains a mutation or a different sequence or an additional nucleotide, it too is incorporated into the newly synthesized strand as a result of the recombination. As a result of the proofreading function, it is possible for the new sequence of DNA to serve as the template. Thus, the transferred DNA is incorporated into the genome.

Attached to these pieces of targeting DNA are regions of DNA which may interact with or control the expression of a polypeptide(s) identified by the methods of the invention, e.g., flanking sequences. For example, a promoter/enhancer element, a suppressor, or an exogenous transcription modulatory element is inserted in the genome of the intended host cell in proximity and orientation sufficient to influence the transcription of DNA encoding the desired polypeptide. The control element controls a portion of the DNA present in the host cell genome. Thus, the expression of the desired polypeptide(s) identified by the methods of the invention may be achieved, not by transfection of DNA that encodes the gene itself, but rather by the use of targeting DNA (containing regions of homology with the endogenous gene of interest) coupled with DNA regulatory segments that provide the endogenous gene sequence with recognizable signals for transcription of a polypeptide(s) identified by the methods of the invention.

In an exemplary method, the expression of a desired targeted gene in a cell (i.e., a desired endogenous cellular gene) is altered via homologous recombination into the cellular genome at a preselected site, by the introduction of DNA which includes at least a regulatory sequence, an exon and a splice donor site. These components are introduced into the chromosomal (genomic) DNA in such a manner that this, in effect, results in the production of a new transcription unit (in which the regulatory sequence, the exon and the splice donor site present in the DNA construct are operatively linked to the endogenous gene). As a result of the introduction of these components into the chromosomal DNA, the expression of the desired endogenous gene is altered.

Altered gene expression, as described herein, encompasses activating (or causing to be expressed) a gene which is normally silent (unexpressed) in the cell as obtained, as well as increasing the expression of a gene which is not expressed at physiologically significant levels in the cell as obtained. The embodiments further encompass changing the pattern of regulation or induction such that it is different from the pattern of regulation or induction that occurs in the cell as obtained, and reducing (including eliminating) the expression of a gene which is expressed in the cell as obtained.

One method by which homologous recombination can be used to increase, or cause, polypeptide production from a cell's endogenous gene involves first using homologous recombination to place a recombination sequence from a site-specific recombination system (e.g., Cre/loxP, FLP/FRT) (Sauer et al., Current Opinion In Biotechnology 5:521-527, 1994; Sauer et al., Methods In Enzymology 225:890-900, 1993) upstream (that is, 5′ to) of the cell's endogenous genomic polypeptide coding region. A plasmid containing a recombination site homologous to the site that was placed just upstream of the genomic polypeptide coding region is introduced into the modified cell line along with the appropriate recombinase enzyme. This recombinase causes the plasmid to integrate, via the plasmid's recombination site, into the recombination site located just upstream of the genomic polypeptide coding region in the cell line (Baubonis et al., Nucleic Acids Res. 21:2025-2029, 1993; O'Gorman et al., Science 251:1351-1355, 1991). Any flanking sequences known to increase transcription (e.g., enhancer/promoter, intron, translational enhancer), if properly positioned in this plasmid, would integrate in such a manner as to create a new or modified transcriptional unit resulting in de novo or increased production of polypeptide(s) identified by the methods of the invention from the cell's endogenous genes.

A further method to use the cell line in which the site specific recombination sequence had been placed just upstream of the cell's endogenous genomic polypeptide coding region is to use homologous recombination to introduce a second recombination site elsewhere in the cell line's genome. The appropriate recombinase enzyme is then introduced into the two-recombination-site cell line, causing a recombination event (deletion, inversion, translocation) (Sauer et al., Current Opinion In Biotechnology, supra, 1994; Sauer, Methods Enzymol., supra, 1993) that would create a new or modified transcriptional unit resulting in de novo or increased polypeptide production from the cell's endogenous gene.

An additional approach for increasing, or causing, the expression of polypeptide(s) identified by the methods of the invention from a cell's endogenous gene(s) involves increasing, or causing, the expression of a gene or genes (e.g., transcription factors) and/or decreasing the expression of a gene or genes (e.g., transcriptional repressors) in a manner which results in de novo or increased polypeptide production from the cell's endogenous gene(s). This method includes the introduction of a non-naturally occurring polypeptide (e.g., a polypeptide comprising a site specific DNA binding domain fused to a transcriptional factor domain) into the cell such that de novo or increased polypeptide production from the cell's endogenous gene(s) results.

The present invention further relates to DNA constructs useful in the method of altering expression of a target gene. In certain embodiments, the exemplary DNA constructs comprise: (a) one or more targeting sequences; (b) a regulatory sequence; (c) an exon; and (d) an unpaired splice-donor site. The targeting sequence in the DNA construct directs the integration of elements (a)-(d) into a target gene in a cell such that the elements (b)-(d) are operatively linked to sequences of the endogenous target gene. In another embodiment, the DNA constructs comprise: (a) one or more targeting sequences, (b) a regulatory sequence, (c) an exon, (d) a splice-donor site, (e) an intron, and (f) a splice-acceptor site, wherein the targeting sequence directs the integration of elements (a)-(f) such that the elements of (b)-(f) are operatively linked to the endogenous gene. The targeting sequence is homologous to the preselected site in the cellular chromosomal DNA with which homologous recombination is to occur. In the construct, the exon is generally 3′ of the regulatory sequence and the splice-donor site is 3′ of the exon.

If the sequence of a particular gene is known, such as the nucleic acid sequence of a polypeptide(s) identified by the methods of the invention presented herein, a piece of DNA that is complementary to a selected region of the gene can be synthesized or otherwise obtained, such as by appropriate restriction of the native DNA at specific recognition sites bounding the region of interest. This piece serves as a targeting sequence(s) upon insertion into the cell and will hybridize to its homologous region within the genome. If this hybridization occurs during DNA replication, this piece of DNA, and any additional sequence attached thereto, will act as an Okazaki fragment and will be incorporated into the newly synthesized daughter strand of DNA. The present invention, therefore, includes nucleotides encoding polypeptide(s) identified by the methods of the invention, which nucleotides may be used as targeting sequences.

Polypeptide cell therapy, e.g., the implantation of cells producing polypeptide(s) identified by the methods of the invention, is also contemplated. This embodiment involves implanting cells capable of synthesizing and secreting a biologically active form of a polypeptide(s) identified by the methods of the invention. Such polypeptide-producing cells can be cells that are natural producers of said polypeptides or may be recombinant cells whose ability to produce polypeptides has been augmented by transformation with a gene encoding the desired polypeptide(s) identified by the methods of the invention or with a gene augmenting the expression of said polypeptide. Such a modification may be accomplished by means of a vector suitable for delivering the gene as well as promoting its expression and secretion. In order to minimize a potential immunological reaction in patients being administered a polypeptide(s) identified by the methods of the invention, as may occur with the administration of a polypeptide of a foreign species, it is preferred that the natural cells producing the polypeptide(s) identified by the methods of the invention be of human origin and produce human polypeptide. Likewise, it is preferred that the recombinant cells producing polypeptide(s) identified by the methods of the invention be transformed with an expression vector containing a gene encoding a human polypeptide.

Implanted cells may be encapsulated to avoid the infiltration of surrounding tissue. Human or non-human animal cells may be implanted in patients in biocompatible, semipermeable polymeric enclosures or membranes that allow the release of polypeptide(s) identified by the methods of the invention, but prevent the destruction of the cells by the patient's immune system or by other detrimental factors from the surrounding tissue. Alternatively, the patient's own cells, transformed to produce polypeptide(s) identified by the methods of the invention ex vivo, may be implanted directly into the patient without such encapsulation.

Techniques for the encapsulation of living cells are known in the art, and the preparation of the encapsulated cells and their implantation in patients may be routinely accomplished. For example, Baetge et al. (WO95/05452; PCT/US94/09299) describe membrane capsules containing genetically engineered cells for the effective delivery of biologically active molecules. The capsules are biocompatible and are easily retrievable. The capsules encapsulate cells transfected with recombinant DNA molecules comprising DNA sequences coding for biologically active molecules operatively linked to promoters that are not subject to down-regulation in vivo upon implantation into a mammalian host. The devices provide for delivery of the molecules from living cells to specific sites within a recipient. In addition, see U.S. Pat. Nos. 4,892,538, 5,011,472, and 5,106,627. A system for encapsulating living cells is described in PCT Application no. PCT/US91/00157 of Aebischer et al. See also, PCT Application No. PCT/US91/00155 of Aebischer et al., Winn et al., Exper. Neurol. 113: 322-329, 1991, Aebischer et al., Exper. Neurol. 111:269-275, 1991; and Tresco et al., ASAIO 38:17-23, 1992.

In vivo and in vitro gene therapy delivery of polypeptide(s) identified by the methods of the invention are also envisioned. One example of a gene therapy technique is to use the gene (either genomic DNA, cDNA, and/or synthetic DNA) encoding a polypeptide(s) identified by the methods of the invention which may be operably linked to a constitutive or inducible promoter to form a “gene therapy DNA construct”. The promoter may be homologous or heterologous to the endogenous gene, provided that it is active in the cell or tissue type into which the construct will be inserted. Other components of the gene therapy DNA construct may optionally include, DNA molecules designed for site-specific integration (e.g., endogenous sequences useful for homologous recombination), tissue-specific promoter, enhancer(s) or silencer(s), DNA molecules capable of providing a selective advantage over the parent cell, DNA molecules useful as labels to identify transformed cells, negative selection systems, cell specific binding agents (as, for example, for cell targeting), cell-specific internalization factors, and transcription factors to enhance expression by a vector as well as factors to enable vector manufacture.

A gene therapy DNA construct can then be introduced into cells (either ex vivo or in vivo) using viral or non-viral vectors. One means for introducing the gene therapy DNA construct is by means of viral vectors as described herein. Certain vectors, such as retroviral vectors, will deliver the DNA construct to the chromosomal DNA of the cells, and the gene can integrate into the chromosomal DNA. Other vectors will function as episomes, and the gene therapy DNA construct will remain in the cytoplasm.

In yet other embodiments, regulatory elements can be included for the controlled expression of the identified gene in the target cell. Such elements are turned on in response to an appropriate effector. In this way, a therapeutic polypeptide can be expressed when desired. One conventional control means involves the use of small molecule dimerizers or rapalogs (as described in WO9641865 (PCT/US96/099486); WO9731898 (PCT/US97/03137) and WO9731899 (PCT/US95/03157) used to dimerize chimeric proteins which contain a small molecule-binding domain and a domain capable of initiating biological process, such as a DNA-binding protein or transcriptional activation protein. The dimerization of the proteins can be used to initiate transcription of the transgene.

An alternative regulation technology uses a method of storing proteins expressed from the gene of interest inside the cell as an aggregate or cluster. The gene of interest is expressed as a fusion protein that includes a conditional aggregation domain which results in the retention of the aggregated protein in the endoplasmic reticulum. The stored proteins are stable and inactive inside the cell. The proteins can be released, however, by administering a drug (e.g., small molecule ligand) that removes the conditional aggregation domain and thereby specifically breaks apart the aggregates or clusters so that the proteins may be secreted from the cell. See, Science 287:816-817, and 826-830 (2000).

Other suitable control means or gene switches include, but are not limited to, the following systems. Mifepristone (RU486) is used as a progesterone antagonist. The binding of a modified progesterone receptor ligand-binding domain to the progesterone antagonist activates transcription by forming a dimer of two transcription factors which then pass into the nucleus to bind DNA. The ligand-binding domain is modified to eliminate the ability of the receptor to bind to the natural ligand. The modified steroid hormone receptor system is further described in U.S. Pat. No. 5,364,791; WO9640911; and WO9710337.

Yet another control system uses ecdysone (a fruit fly steroid hormone) which binds to and activates an ecdysone receptor (cytoplasmic receptor). The receptor then translocates to the nucleus to bind a specific DNA response element (promoter from ecdysone-responsive gene). The ecdysone receptor includes a transactivation domain/DNA-binding domain/ligand-binding domain to initiate transcription. The ecdysone system is further described in U.S. Pat. No. 5,514,578; WO9738117; WO9637609; and WO9303162.

Another control means uses a positive tetracycline-controllable transactivator. This system involves a mutated tet repressor protein DNA-binding domain (mutated tet R-4 amino acid changes which resulted in a reverse tetracycline-regulated transactivator protein, i.e., it binds to a tet operator in the presence of tetracycline) linked to a polypeptide which activates transcription. Such systems are described in U.S. Pat. Nos. 5,464,758; 5,650,298 and 5,654,168.

Additional expression control systems and nucleic acid constructs are described in U.S. Pat. Nos. 5,741,679 and 5,834,186, to Innovir Laboratories Inc.

In vivo gene therapy may be accomplished by introducing the gene encoding a polypeptide(s) identified by the methods of the invention into cells via local injection of an identified polypeptide's encoding nucleic acid molecule or by other appropriate viral or non-viral delivery vectors. (Hefti, Neurobiology 25:1418-1435, 1994). For example, a nucleic acid molecule encoding a polypeptide may be contained in an adeno-associated virus (AAV) vector for delivery to the targeted cells (e.g., Johnson, International Publication No. WO95/34670; International Application No. PCT/US95/07178). The recombinant AAV genome typically contains AAV inverted terminal repeats flanking a DNA sequence encoding a polypeptide(s) identified by the methods of the invention operably linked to functional promoter and polyadenylation sequences.

Alternative suitable viral vectors include, but are not limited to, retrovirus, adenovirus, herpes simplex virus, lentivirus, hepatitis virus, parvovirus, papovavirus, poxvirus, alphavirus, coronavirus, rhabdovirus, paramyxovirus, and papilloma virus vectors. U.S. Pat. No. 5,672,344 describes an in vivo viral-mediated gene transfer system involving a recombinant neurotrophic HSV-1 vector. U.S. Pat. No. 5,399,346 provides examples of a process for providing a patient with a therapeutic protein by the delivery of human cells which have been treated in vitro to insert a DNA segment encoding a therapeutic protein. Additional methods and materials for the practice of gene therapy techniques are described in U.S. Pat. No. 5,631,236 involving adenoviral vectors; U.S. Pat. No. 5,672,510 involving retroviral vectors; and U.S. Pat. No. 5,635,399 involving retroviral vectors expressing cytokines.

Nonviral delivery methods include, but are not limited to, liposome-mediated transfer, naked DNA delivery (direct injection), receptor-mediated transfer (ligand-DNA complex), electroporation, calcium phosphate precipitation, and microparticle bombardment (e.g., gene gun). Gene therapy materials and methods may also include the use of inducible promoters, tissue-specific enhancer-promoters, DNA sequences designed for site-specific integration, DNA sequences capable of providing a selective advantage over the parent cell, labels to identify transformed cells, negative selection systems and expression control systems (safety measures), cell-specific binding agents (for cell targeting), cell-specific internalization factors, and transcription factors to enhance expression by a vector as well as methods of vector manufacture. Such additional methods and materials for the practice of gene therapy techniques are described in U.S. Pat. No. 4,970,154 involving electroporation techniques; WO96/40958 involving nuclear ligands; U.S. Pat. No. 5,679,559 describing a lipoprotein-containing system for gene delivery; U.S. Pat. No. 5,676,954 involving liposome carriers; U.S. Pat. No. 5,593,875 concerning methods for calcium phosphate transfection; and U.S. Pat. No. 4,945,050 wherein biologically active particles are propelled at cells at a speed whereby the particles penetrate the surface of the cells and become incorporated into the interior of the cells.

It is also contemplated that gene therapy or cell therapy can further include the delivery of one or more additional polypeptide(s) in the same or a different cell(s). Such cells may be separately introduced into the patient, or the cells may be contained in a single implantable device, such as the encapsulating membrane described above, or the cells may be separately modified by means of viral vectors.

A means to increase endogenous expression of a polypeptide(s) identified by the methods of the invention in a cell via gene therapy is to insert one or more enhancer element(s) into the polypeptide promoter, where the enhancer element(s) can serve to increase transcriptional activity of the gene. The enhancer element(s) used will be selected based on the tissue in which one desires to activate the gene(s); enhancer elements known to confer promoter activation in that tissue will be selected. For example, if a gene encoding a polypeptide(s) identified by the methods of the invention is to be “turned on” in T-cells, the Ick promoter enhancer element may be used. Here, the functional portion of the transcriptional element to be added may be inserted into a fragment of DNA containing the identified polypeptide promoter (and optionally, inserted into a vector and/or 5′ and/or 3′ flanking sequence(s), etc.) using standard cloning techniques. This construct, known as a “homologous recombination construct”, can then be introduced into the desired cells either ex vivo or in vivo.

Gene therapy also can be used to decrease polypeptide expression by modifying the nucleotide sequence of the endogenous promoter(s). Such modification is typically accomplished via homologous recombination methods. For example, a DNA molecule containing all or a portion of the promoter of the gene(s) selected for inactivation can be engineered to remove and/or replace pieces of the promoter that regulate transcription. For example the TATA box and/or the binding site of a transcriptional activator of the promoter may be deleted using standard molecular biology techniques; such deletion can inhibit promoter activity thereby repressing the transcription of the corresponding gene. The deletion of the TATA box or the transcription activator binding site in the promoter may be accomplished by generating a DNA construct comprising all or the relevant portion of the polypeptide promoter(s) (from the same or a related species as the gene(s) to be regulated) in which one or more of the TATA box and/or transcriptional activator binding site nucleotides are mutated via substitution, deletion and/or insertion of one or more nucleotides. As a result, the TATA box and/or activator binding site has decreased activity or is rendered completely inactive. The construct will typically contain at least about 500 bases of DNA that correspond to the native (endogenous) 5′ and 3′ DNA sequences adjacent to the promoter segment that has been modified. The construct may be introduced into the appropriate cells (either ex vivo or in vivo) either directly or via a viral vector as described herein. Typically, the integration of the construct into the genomic DNA of the cells will be via homologous recombination, where the 5′ and 3′ DNA sequences in the promoter construct can serve to help integrate the modified promoter region via hybridization to the endogenous chromosomal DNA.

### J. PHARMACEUTICAL COMPOSITIONS AND ADMINISTRATION

Pharmaceutical compositions are within the scope of the present invention. Such pharmaceutical compositions may comprise a therapeutically effective amount of an identified agonist or antagonist/inhibitor in admixture with a pharmaceutically or physiologically acceptable formulation agent selected for suitability with the mode of administration. Likewise, they may also comprise a therapeutically effective amount of one or more selective binding agents in admixture with a pharmaceutically or physiologically acceptable formulation agent selected for suitability with the mode of administration. Such compositions may be administered in therapeutically effective amounts depending on the application.

Acceptable formulation materials preferably are nontoxic to recipients at the dosages and concentrations employed.

The pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

The optimal pharmaceutical composition will be determined by one skilled in the art depending upon, for example, the intended route of administration, delivery format, and desired dosage. See for example, Remington's Pharmaceutical Sciences, supra. Such compositions may influence the physical state, stability, rate of in vivo release, and rate of in vivo clearance of the molecule.

The primary vehicle or carrier in a pharmaceutical composition may be either aqueous or non-aqueous in nature. For example, a suitable vehicle or carrier may be water for injection, physiological saline solution or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which may further include sorbitol or a suitable substitute therefor. In one embodiment of the present invention, polypeptide compositions may be prepared for storage by mixing the selected composition having the desired degree of purity with optional formulation agents (Remington's Pharmaceutical Sciences, supra) in the form of a lyophilized cake or an aqueous solution. Further, the polypeptide product may be formulated as a lyophilizate using appropriate excipients such as sucrose.

Pharmaceutical compositions can be selected for parenteral delivery. Alternatively, the compositions may be selected for inhalation or for delivery through the digestive tract, such as orally. The preparation of such pharmaceutically acceptable compositions is within the skill of the art.

The formulation components are present in concentrations that are acceptable to the site of administration. For example, buffers are used to maintain the composition at physiological pH or at slightly lower pH, typically within a pH range of from about 5 to about 8.

When parenteral administration is contemplated, the therapeutic compositions for use in this invention may be in the form of a pyrogen-free, parenterally acceptable aqueous solution comprising the desired agonist or antagonist/inhibitor molecule in a pharmaceutically acceptable vehicle. A particularly suitable vehicle for parenteral injection is sterile distilled water in which an agonist or antagonist/inhibitor molecule is formulated as a sterile, isotonic solution, properly preserved. Yet another preparation can involve the formulation of the desired molecule with an agent, such as injectable microspheres, bio-erodible particles, polymeric compounds (polylactic acid, polyglycolic acid), beads, or liposomes, which provides for the controlled or sustained release of the product which may then be delivered via a depot injection. Hyaluronic acid may also be used, and this may have the effect of promoting sustained duration in the circulation. Other suitable means for the introduction of the desired molecule include implantable drug delivery devices.

In one embodiment, a pharmaceutical composition may be formulated for inhalation. For example, an agonist or antagonist/inhibitor molecule may be formulated as a dry powder for inhalation. Agonist or antagonist/inhibitor molecule inhalation solutions may also be formulated with a propellant for aerosol delivery. In yet another embodiment, solutions may be nebulized. Pulmonary administration is further described in PCT Application No. PCT/US94/001875, which describes pulmonary delivery of chemically modified proteins.

It is also contemplated that certain formulations may be administered orally. In one embodiment of the present invention, agonist or antagonist/inhibitor molecules which are administered in this fashion can be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents can be included to facilitate absorption of the agonist or antagonist/inhibitor molecule. Diluents, flavorings, low melting point waxes, vegetable oils, lubricants, suspending agents, tablet disintegrating agents, and binders may also be employed.

Another pharmaceutical composition may involve an effective quantity of an agonist or antagonist/inhibitor molecule in a mixture with non-toxic excipients which are suitable for the manufacture of tablets. By dissolving the tablets in sterile water, or other appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving identified agonist or antagonist/inhibitor molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See for example, PCT/US93/00829 which describes controlled release of porous polymeric microparticles for the delivery of pharmaceutical compositions. Additional examples of sustained-release preparations include semipermeable polymer matrices in the form of shaped articles, e.g. films, or microcapsules. Sustained release matrices may include polyesters, hydrogels, polylactides (U.S. Pat. No. 3,773,919, EP 58,481), copolymers of L-glutamic acid and gamma ethyl-L-glutamate (Sidman et al., Biopolymers, 22:547-556, 1983), poly(2-hydroxyethyl-methacrylate) (Langer et al., J. Biomed. Mater. Res., 15:167-277, 1981; Langer et al., Chem. Tech. 12:98-105,1982), ethylene vinyl acetate (Langer et al., supra) or poly-D(−)-3-hydroxybutyric acid (EP 133,988). Sustained-release compositions also include liposomes, which can be prepared by any of several methods known in the art. See e.g., Eppstein et al., Proc. Natl. Acad. Sci. USA, 82:3688-3692, 1985; EP 36,676; EP 88,046; EP 143,949. Formulations that facilitate delivery to the CNS are preferred for this invention.

The pharmaceutical composition to be used for in vivo administration typically must be sterile. This may be accomplished by filtration through sterile filtration membranes. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

Once the pharmaceutical composition has been formulated, it may be stored in sterile vials as a solution, suspension, gel, emulsion, solid, or a dehydrated or lyophilized powder. Such formulations may be stored either in a ready-to-use form or in a form (e.g., lyophilized) requiring reconstitution prior to administration.

In a specific embodiment, the present invention is directed to kits for producing a single-dose administration unit. The kits may each contain both a first container having a dried protein and a second container having an aqueous formulation. Also included within the scope of this invention are kits containing single and multi-chambered pre-filled syringes (e.g., liquid syringes and lyosyringes).

An effective amount of a pharmaceutical composition to be employed therapeutically including, but not limited to, the treatment of neuronal injury or neurodegeneration will depend, for example, upon the therapeutic context and objectives. One skilled in the art will appreciate that the appropriate dosage levels for treatment will thus vary depending, in part, upon the molecule delivered, the indication for which the agonist or antagonist/inhibitor molecule is being used, the route of administration, and the size (body weight, body surface or organ size) and condition (the age and general health) of the patient. Accordingly, the clinician may titer the dosage and modify the route of administration to obtain the optimal therapeutic effect. A typical dosage may range from about 0.01 μg/kg to up to about 100 mg/kg or more, depending on the factors mentioned above. In other embodiments, the dosage may range from 0.1 mg/kg up to about 100 mg/kg; or 1 mg/kg up to about 100 mg/kg; or 5 mg/kg up to about 100 mg/kg.

The frequency of dosing will depend upon the pharmacokinetic parameters of the agonist or antagonist/inhibitor molecule in the formulation used. Typically, a clinician will administer the composition until a dosage is reached that achieves the desired effect. The composition may therefore be administered as a single dose, or as two or more doses (which may or may not contain the same amount of the desired molecule) over time, or as a continuous infusion via implantation device or catheter. Further refinement of the appropriate dosage is routinely made by those of ordinary skill in the art and is within the ambit of tasks routinely performed by them. Appropriate dosages may be ascertained through use of appropriate dose-response data.

The route of administration of the pharmaceutical composition is in accord with known methods, e.g. orally, through injection by intravenous, intraperitoneal, intracerebral (intra-parenchymal), intracerebroventricular, intracranial, intramuscular, intra-ocular, intraarterial, intraportal, or intralesional routes, by sustained release systems or by implantation devices. Where desired, the compositions may be administered by bolus injection or continuously by infusion, or by implantation device.

Alternatively or additionally, the composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed-release bolus, or continuous administration.

In some cases, it may be desirable to use pharmaceutical compositions in an ex vivo manner. In such instances, cells, tissues, or organs that have been removed from the patient are exposed to pharmaceutical compositions after which the cells, tissues and/or organs are subsequently implanted back into the patient.

In other cases, an agonist or antagonist/inhibitor molecule can be delivered by implanting certain cells that have been genetically engineered, using methods such as those described herein, to express and secrete the polypeptide of the identified agonist or antagonist/inhibitor. Such cells may be animal or human cells, and may be autologous, heterologous, or xenogeneic. Optionally, the cells may be immortalized. In order to decrease the chance of an immunological response, the cells may be encapsulated to avoid infiltration of surrounding tissues. The encapsulation materials are typically biocompatible, semi-permeable polymeric enclosures or membranes that allow the release of the protein product(s) but prevent the destruction of the cells by the patient's immune system or by other detrimental factors from the surrounding tissues.

### K. THERAPEUTIC USES OF THE IDENTIFIED AGONISTS AND ANTAGONISTS/INHIBITORS

Methods for the treatment of neural injury or neurodegeneration using pharmaceutical compositions of the invention are within the scope of the present invention. More specifically, the present invention includes the use of pharmaceutical compositions in neurogenesis and neuroregeneration (or in the manufacture of medicaments for treatment of conditions that benefit from neurogenesis or neuroregeneration). To understand how such compositions might affect these processes, it is important to understand some aspects of neuroregeneration, neurogenesis, and neurodegenerative diseases and disorders as set out below.

1. CNS Regeneration or Neuroregeneration and Neurogenesis

CNS regeneration or neuroregeneration refers to one of several types of events that lead to functional improvement of the CNS which can manifest itself as an improvement, stabilization, or slowed-down deterioration of functions such as cognition, vision, etc. The cellular basis for CNS regeneration (neuroregeneration) can be e.g. increased proliferation (or slower death) of neural stem cells, generation of new neurons and/or glial cells (referred to as neurogenesis and gliogenesis, respectively), formation of new, or stabilization of the existing, neuronal synapses (synaptic regeneration), or re-growth of severed axons (axonal regeneration), etc.

2. CNS Regeneration and the Treatment of CNS Trauma and Neuorodegenerative Diseases and Disorders

CNS regeneration is a desirable event that can slow down the progression or ameliorate consequences of many of the conditions that negatively affect the CNS and often lead to the loss of neuronal, astroglial, and other cells or causes their functional impairment. Such conditions include CNS trauma, ischemic and hypoxic damage, toxic damage, damage connected with a metabolic impairment (e.g. diabetes and diabetic retinopathy), the whole range of neurodegenerative diseases (e.g. Alzheimer's disease, Parkinson's disease, multiple sclerosis, Jacob-Creutzfelt's disease and other prion diseases), epilepsy as well as aging. The latter is not considered a disease (since it is inevitable and affects all individuals) but is associated with the same loss of neuronal and glial cells and deterioration of their function, albeit more discrete and with a comparably slow progression.

3. Astrocytes and the Fate of Neural Transplants

With recent progress in neuroscience and stem cell research, neural transplantation has emerged as a promising therapy for selected diseases of the CNS. However, the success of transplantation has been limited by the restricted ability of neural implants to survive, integrate, and establish neuronal connections with the host environment. Most grafted cells die, and the majority of those that survive do not migrate out of the injection site and lack axon- or dendrite-like processes that extend into the host. Since immature neurons and neural stem cells, in general, are intrinsically capable of migrating, differentiating, and growing neurites, it is possible that the normal CNS environment presents a barrier to graft integration and formation of synaptic function. Thus, active agents identified in the methods of the invention can be used to improve survival, migration, differentiation and/or stable integration in the CNS, i.e. brain, spinal cord or retina.

### L. EXAMPLES

The candidate modulators identified by the initial screens are evaluated for their effect on neurogenesis and neuroregeneration using in vivo or ex vivo (cell-free, cell culture, organ, etc.) systems. Other aspects and advantages of the present invention will be understood upon consideration of the following illustrative examples. Those of skill in the art will, in light of the present disclosure, appreciate that many changes can be made in the specific materials and methods which are disclosed and still obtain a like or similar result without departing from the spirit and scope of the invention.

Example 1 describes the identification of molecular targets by DNA arrays and protein analysis; Example 2 provides a screening assay for complement 3a receptor (C3aR) or complement 5a receptor (C5aR) agonists; Example 3 discloses a screening assay for inhibitors of IGFBP-4; Example 4 describes a screening assay for inhibitors of UNC-51-like kinase (ULK); Example 5 provides a screening assay for effect(s) of endothelin receptor B (ETBR) antagonists in neurons, neuronal stem cells, and in animals after neural injury; Example 6 describes entorhinal cortex lesion as a means of evaluating neuroregeneration; Example 7 discloses neuronal cell cultures; Example 8 discusses evaluation of astrocytic processes by immunohistochemistry; Example 9 describes dye filling of astrocytes; Example 10 provides a method for the quantification of proliferating cells in the brain; Example 11 describes the quantification of newly generated neurons in the brain; Example 12 describes methods of evaluating cognitive function in mice; Example 13 provides an assay of optic nerve injury and treatment with modulators of neuroregeneration; Example 14 discloses C3aR and C5aR expression on neural stem cells and neural progenitor cells; Example 15 discusses reduced basal neurogenesis in mice treated with a C3aR antagonist; Example 16 reports a reduced number of neuroblasts in the SVZ in the injured hemisphere of C3−/− mice after brain ischemia; Example 17 discloses that C3−/− and control mice respond to brain ischemia by increased cell proliferation in the ipsilateral SVZ; Example 18 describes that C3−/− mice have fewer proliferating nonmicroglial/nonendothelial cells in the prenumbra than controls; Example 19 discloses that C3−/− mice have fewer neural progenitor cells in the prenumbra and infarct area than controls; Example 20 demonstrates that C3−/− mice have fewer newly-formed neurons after ischemia than controls; Example 21 discloses larger infarcts in C3−/− mice than in controls; Example 22 demonstrates that the activation of astrocytes and microglia at the infarct border is similar in C3−/− and control mice; and Example 23 demonstrates the importance of using a neural cell line to test the effects of C3aR agonists and antagonists on neural stem cell differentiation.

The section headings used herein are for organizational purposes only and are not to be construed as limiting the subject matter described therein. All references cited in this application are expressly incorporated by reference herein.

### Example 1

### Identification of Molecular Targets by DNA Arrays and Protein Analysis

DNA arrays, PCR (including quantitative RT-PCR), and subsequent protein analyses were used as a means to characterize gene and protein expression levels in normal mice and in the regeneration-supporting environment in GFAP−/−Vim−/− mice, to identify molecular targets that promote neurogenesis and regeneration in the adult CNS.

Transgenic mice with the IF proteins GFAP and vimentin genetically ablated (Eliasson et al. J. Biol. Chem. 274:23996-4006., 1999), were used as a unique mammalian model of enhanced neuroregeneration (Kinouchi et al., Nat Neurosci. 6:863-8, 2003). DNA array technology was used to compare the gene expression profiles between wild-type (standard, regeneration-inhibiting) and GFAP−/−Vim−/− (regeneration-supporting) environments. Four experimental systems were used: (a) postnatal (P1) day brains, (b) astrocyte-enriched cultures prepared from P1 brains, (c) adult brains, and (d) the brain tissue surrounding cortical brain lesions (electrically induced, as described in Enge et al., Neurochem Res. 28:271-279, 2003).

Approximately 50 genes were found to be differentially expressed (2-fold or greater) between wild-type and knockout (GFAP−/−Vim−/−) mice in one or several of the systems as set out above. A number of these differentially expressed genes were further studied by PCR and/or immunohistochemical/biochemical analysis of their respective encoded proteins. These differentially expressed genes/proteins were selected as targets for modulation, to promote regeneration in the CNS, and are discussed in further detail below.

### Example 2

### Screening Assay for C3a Receptor (C3aR) or C5a Receptor (C5aR) Agonists

Neural stem cells, migrating neuroblasts, immature neurons, and mature neurons all express receptors for complement fragments C3a and C5a. After permanent focal ischemia, mice deficient in the third complement component (C3 −/−) had 30-50% fewer neural progenitor cells and 25% fewer newly formed neurons in the prenumbra than controls. C3−/− mice also had larger infarct volumes and 24% fewer migrating neural progenitor cells in the subventricular zone. Thus, there is evidence that the complement system promotes neuroregeneration after cerebral ischemia via signaling through the complement 3a receptor (C3aR) and/or the complement 5a receptor (C5aR). The present invention includes a method of screening for C3aR or C5aR agonists.

Cells, e.g., rat basophilic leukemia cell line, RBL-2H3 (ATCC no. CRL-2256) stably expressing human C3aR (polynucleotide, SEQ ID NO: 4; polypeptide, SEQ ID NO: 5) or human C5aR (polynucleotide, SEQ ID NO: 11; polypeptide, SEQ ID NO: 12), are maintained under standard cell culture conditions in Eagle's MEM with Earle's salts, with L-glutamine and nonessential amino acids supplemented with FBS (10%) and G418 (400 μg/ml). A radioligand binding assay, based on the use of C3aR- or C5aR-expressing cells, or membranes of cells thereof, and ¹²⁵I-labelled human C3a (polynucleotide, SEQ ID NO: 1; polypeptide, SEQ ID NO: 2) or ¹²⁵I-labelled human C5a (polynucleotide, SEQ ID NO: 8; polypeptide, SEQ ID NO: 9), respectively, is performed in a 96-well microtiter plate format. Live cells, or membranes thereof, are bound to beads. Each plate well contains either ¹²⁵I-C3a or ¹²⁵I-labelled C5a in binding buffer. Control wells, used to measure nonspecific binding, include an excess of unlabeled C3a or C5a. Putative modulators of C3aR or C5aR, such as non-peptide (e.g., small molecules from chemical libraries), or peptide, compounds are added. After incubation, the plates are washed and the plate-bound radioactivity is counted on a scintillation counter. The greater the binding of a test compound to the receptor (indicative of possible agonist or antagonist activity), the less of the radiolabelled ligand will bind to the cells or the cell membranes bound to the plate, which in turn results in a lower radioactivity count.

Compounds with high affinity binding to the respective receptor are further tested for an agonistic effect in another assay to distinguish agonists from antagonists, e.g., in a receptor internalization assay, a chemotaxis assay, a H₂O₂ production assay, a cell proliferation assay, a cell differentiation assay, and/or in an animal model of stroke or neural injury, etc. Exemplary assays are described herein.

In one variation, A receptor internalization assay uses human neutrophils which are stimulated by incubation with the natural ligands C3a or C5a, or with putative agonist compounds. C3aR or C5aR are detected by polyclonal antibodies, and receptor internalization is quantified by flow cytometry. Increased receptor internalization correlates with reduced levels of receptors detected on the cell surface. Compounds that induce an effect similar to, or greater than, the natural ligand(s) are regarded as agonists.

A chemotaxis assay uses human neutrophils which are fluorescein-labeled with 2′,7′-bis-(2-carboxyethyl)-5-(and 6)-carboxyfluorescein acetoxymethyl ester) for 30 min at 37° C. After a washing step, labeled neutrophils (5×10⁶ cells/ml) are loaded into the upper chamber of a 96-well mini chamber, separated by a polycarbonate filter with a porosity of 3 μm. Lower chambers are loaded with different concentrations of C3a polypeptide, C5a polypeptide, or putative agonist compounds. Cells are incubated for 30 min at 37° C., and the number of cells that migrate through the polycarbonate membrane to the lower surface is determined by cytofluorometry.

A H₂O₂ production assay determines H₂O₂ production by isolated human neutrophils. In this assay, cells are pretreated for 1 h at 37° C. with either C3a polypeptide or C5a polypeptide (@ 10 nM) or a putative agonist compound(s) in the presence of 1 mM sodium azide (to prevent endogenous catalases from destroying H₂O₂). To stimulate neutrophils, phorbol myristate acetate (25 ng/ml) is added at the end of the preincubation and cell suspensions are incubated at 37° C. for 15 min. The reaction is terminated by the addition of 0.1 ml of trichloroacetic acid (50% w/v). Samples are then centrifuged for 10 min at 500×g, and ferrous ammonium sulfate and potassium thiocyanate are added to the supernatant at final concentrations of 1.5 mM and 0.25 M, respectively. The absorbance of the ferrithiocyanate complex is measured at 480 nm and is compared with a standard curve generated from dilutions of reference solutions of H₂O₂.

A compound(s) identified as a putative C3aR or C5aR agonist(s) is also tested for its effect(s) on the in vitro proliferation and/or differentiation of neural stem cells, e.g., HCNA 94/GFPH (Palmer et al., Mol. Cell Neurosci. 6:389-404, 1997; Takahashi et al., 1999. J. Neurobiol. 38:65-81, 1999) and immature neural and glial cells. Primordial neural stem cells are also derived from the adult rat hippocampus (Takahashi et al., supra).

The putative agonist(s) is also tested for its effect on neurogenesis in vivo. The agonist(s) is administered orally or via an intraperitoneal, intravenous, intracranial, or subcutaneous injection. The agonist's effect(s) on basal neurogenesis, as well as injury-induced neurogenesis, is determined by BrdU injections and immunostaining for Ki67, BrdU, NeuN, or doublecortin in various animal models of neural injury, e.g., middle cerebral artery occlusion (MCAO), electrical injury, and entorhinal cortex lesion. An increased number of Ki67-, BrdU-, NeuN-, or doublecortin-positive immunostained cells, compared to control (non-treated animals) indicates a positive effect on neurogenesis.

C3a and C5a ligands, fragments thereof, and the C3aR and C5aR agonist compounds, identified by the methods of the invention, are then used to stimulate neurogenesis after cerebral ischemia and other types of brain injury, including neurodegenerative disorders, such as Alzheimer's disease, and to counteract the loss of neurons during aging.

### Example 3

### Screening Assay for IGFBP-4 Antagonists

In the hippocampus of the uninjured control mouse brain, astrocytes were found to be highly positive for IGFBP-4 (SEQ ID NO: 20). Likewise, in the hippocampus of the injured control mouse brain (after entorhinal cortex lesions), reactive astrocytes were found to be highly positive for IGFBP-4. However, both non-reactive and reactive astrocytes in GFAP−/−Vim−/− (knockout) mice, that exhibit a regeneration-permissive environment, are negative for IGFBP-4. These data provide an indication to use IGFBP-4 inhibitors to create a regeneration-permissive environment to stimulate regeneration. The present invention contemplates a method of screening for IGFBP-4 inhibitors/antagonists.

Primary mouse astrocytes or human astrocytoma cell lines, e.g. U-87 (ATCC No. HTB-14) are maintained under standard cell culture conditions in Eagle's MEM with Earle's salts, with L-glutamine and nonessential amino acids supplemented with fetal calf serum (10%). Binding assays are performed in a 96-well microtiter plate format containing live cells. Each well is first coated with anti-IGFBP-4 antibody. Control wells without cells are included for non-specific binding of the secondary antibody. Non-peptide, as well as peptide, compounds (antagonists) are added and the cells are incubated. After incubation, the medium is removed from the wells and the cells are fixed with methanol. Plate-bound/cell-bound IGFBP-4 is detected by the addition of a secondary anti-IGFBP-4 antibody that is either biotinylated or horse radish peroxidase (HRP)-conjugated. Plates containing biotinylated secondary antibody are further incubated with HRP-streptavidin. After development with a color substrate, the intensity of staining is quantified in a spectrophotometer.

Compounds that lead to down-regulation of IGFBP-4 expression (antagonists) exhibit reduced optical density, in comparison with cells incubated without any test compound. Putative antagonists of IGFBP-4 are further tested for cell-toxicity. Non-toxic compounds that induce reduction of optical density in the above assay (down-regulate IGFBP-4 expression) by more than 50% are considered to be IGFBP-4 antagonists. IGFBP-4 antagonists are further tested in vitro and in vivo as set out below.

Astrocyte and neuron cultures from either postnatal day 0-2 mouse brains or postnatal day 6 cerebella are prepared either as explant cultures or by trypsinization (Pekny et al., Exp. Cell Res. 239: 332, 1998) and are allowed to grow into subconfluent state. Astrocytes are then plated on coverslips into 24-well culture plates, or alternatively, directly into 24 or 96-well plates. Assays for neurite outgrowth are performed by culturing neurons on confluent monolayers of astrocytes in DMEM-F12 containing 5% fetal calf or bovine serum and 50 ng/ml NGF and various concentrations of the IGFBP-4 antagonist. After 6-24 hours, the cultures are fixed with 4% paraformaldehyde and immunostaining is perfomed by using antibodies against tubulin. The outgrowth of neurites is assessed from captured images. Neurite length is assessed as the distance from the center of the cell soma to the tip of its longest neurite. A tested substance is considered to have an IGFBP-4 antagonistic effect (neuroregeneration-promoting effect), if in its presence, the average neurite length exceeds the average neurite length in cultures maintained without such a substance.

The IGFBP-4 antagonist(s) is then tested for its effect on neurogenesis in vivo. The putative antagonist compound(s) is administered orally or via an intraperitoneal, intravenous, intracranial, or subcutaneous injection. The antagonist's effect(s) on basal neurogenesis, as well as injury-induced neurogenesis, is determined by BrdU injections and immunostaining for Ki67, BrdU, NeuN, or doublecortin in various animal models of neural injury, e.g., middle cerebral artery occlusion (MCAO), electrical injury, and entorhinal cortex lesion. An increased number of Ki67-, BrdU-, NeuN-, or doublecortin-positive immunostained cells, compared to control (non-treated animals), indicates a positive effect on neurogenesis.

IGFBP-4, fragments thereof, and the putative IGFBP-4 down-regulating compounds (antagonists), identified by the methods of the invention, are then used to stimulate neurogenesis after cerebral ischemia and other types of brain injury, including neurodegenerative disorders, such as Alzheimer's disease, and to counteract the loss of neurons during aging.

### Example 4

### Screening Assay for Inhibitors UNC-51-Like Kinase (ULK)

Primary mouse astrocytes or human astrocytoma cell lines, e.g. U-87 (ATCC no. HTB-14) are maintained under standard cell culture conditions in Eagle's MEM with Earle's salts, with L-glutamine and nonessential amino acids supplemented with fetal calf serum (10%). Binding assays are performed in a 96-well microtiter plate format containing live cells. Each well is first coated with anti-ULK antibody. Control wells without cells are included for non-specific binding of the secondary antibody. Non-peptide, as well as peptide, compounds (antagonists) are added and the cells are incubated. After incubation, the medium is removed from the wells and the cells are fixed with methanol. Plate-bound/cell-bound ULK is detected by the addition of a secondary anti-ULK antibody that is either biotinylated or horse radish peroxidase (HRP)-conjugated. Plates containing biotinylated secondary antibody are further incubated with HRP-streptavidin. After development with a color substrate, the intensity of staining is quantified in a spectrophotometer.

Compounds that lead to down-regulation of ULK expression (antagonists) exhibit reduced optical density, in comparison with cells incubated without any test compound. Putative antagonists of ULK are further tested for cell-toxicity. Non-toxic compounds that induce a reduction in optical density in an assay as set out above (down-regulate ULK expression) by more than 50% are considered to be ULK antagonists. ULK antagonists are further tested in vitro and in vivo as set out below.

Astrocyte and neuron cultures from either postnatal day 0-2 mouse brains or postnatal day 6 cerebella are prepared either as explant cultures or by trypsinization (Pekny et al., Exp. Cell Res. 239: 332, 1998) and are allowed to grow into subconfluent state. Astrocytes are then plated on coverslips into 24-well culture plates, or alternatively, directly into 24 or 96-well plates. Assays for neurite outgrowth are performed by culturing neurons on confluent monolayers of astrocytes in DMEM-F12 containing 5% fetal calf or bovine serum and 50 ng/ml NGF and various concentrations of the ULK antagonist. After 6-24 hours, the cultures are fixed with 4% paraformaldehyde and immunostaining is perfomed by using antibodies against tubulin. The outgrowth of neurites is assessed from captured images. Neurite length is assessed as the distance from the center of the cell soma to the tip of its longest neurite. A tested substance is considered to have an ULK antagonistic effect (neuroregeneration-promoting effect), if in its presence, the average neurite length exceeds the average neurite length in cultures maintained without such a substance.

The ULK antagonist(s) is then tested for its effect on neurogenesis in vivo. The putative antagonist compound(s) is administered orally or via an intraperitoneal, intravenous, intracranial, or subcutaneous injection. The antagonist's effect(s) on basal neurogenesis, as well as injury-induced neurogenesis, is determined by BrdU injections and immunostaining for Ki67, BrdU, NeuN, or doublecortin in various animal models of neural injury, e.g., middle cerebral artery occlusion (MCAO), electrical injury, and entorhinal cortex lesion. An increased number of Ki67-, BrdU-, NeuN-, or doublecortin-positive immunostained cells, compared to control (non-treated animals), indicates a positive effect on neurogenesis.

ULK, fragments thereof, and the putative ULK downregulating compounds (antagonists), identified by the methods of the invention, are then used to stimulate neurogenesis after cerebral ischemia and other types of brain injury, including neurodegenerative disorders, such as Alzheimer's disease, and to counteract the loss of neurons during aging.

### Example 5

### Screening Assay(s) for Effect(s) of Endothelin Receptor B (ETBR) Antagonists in Neurons, Neuronal Stem Cells, and in Animals after Neural Injury

In GFAP−/−Vim−/− knockout mice (regeneration-permissive environment), ETBR protein was undetectable on reactive astrocytes, while very abundant on reactive astrocytes of normal mice. These results therefore suggest that the upregulation or activation of ETBR by reactive astrocytes requires an intermediate filament network, and in the absence of ETBR upregulation or activation, there is improved posttraumatic regeneration. Therefore, the present invention contemplates the use of ETBR antagonists to stimulate neurogenesis or neuroregeneration. Known selective inhibitors (antagonists) of ETBR, such as BQ-788 (Calbiochem) and A-192621 (Abbott Laboratories) (Bagnato et al., Cancer Res. 64:1436-1443, 2004; Rosano et al., Am J Pathol. 163:753-762, 2003), are tested for their effects in vitro and/or in vivo as set out below.

Astrocyte and neuron cultures from either postnatal day 0-2 mouse brains or postnatal day 6 cerebella are prepared either as explant cultures or by trypsinization (Pekny et al., Exp. Cell Res. 239: 332, 1998) and are allowed to grow into subconfluent state. Astrocytes are then plated on coverslips into 24-well culture plates, or alternatively, directly into 24 or 96-well plates. Assays for neurite outgrowth are performed by culturing neurons on confluent monolayers of astrocytes in DMEM-F12 containing 5% fetal calf or bovine serum and 50 ng/ml NGF and various concentrations of the ETBR antagonist. After 6-24 hours, the cultures are fixed with 4% paraformaldehyde and immunostaining is perfomed by using antibodies against tubulin. The outgrowth of neurites is assessed from captured images. Neurite length is assessed as the distance from the center of the cell soma to the tip of its longest neurite. A tested substance is considered to have an antagonistic effect, if in its presence, the average neurite length exceeds the average neurite length in cultures maintained without such a substance.

An ETBR antagonist is also tested for its effect on neurogenesis in vivo. The ETBR antagonist(s) is administered orally or via an intraperitoneal injection intraperitoneal, intravenous, intracranial, or subcutaneous injection. The antagonist's effect(s) on basal neurogenesis, as well as injury-induced neurogenesis, is determined by BrdU injections and immunostaining for Ki67, BrdU, NeuN, or doublecortin in various animal models of neural injury, e.g., middle cerebral artery occlusion (MCAO), electrical injury, and entorhinal cortex lesion. An increased number of Ki67-, BrdU-, NeuN-, or doublecortin-positive immunostained cells, compared to control (non-treated animals), indicates a positive effect on neurogenesis.

ETBR antagonists are then used to stimulate neurogenesis after cerebral ischemia and other types of brain injury, including neurodegenerative disorders, such as Alzheimer's disease, and to counteract the loss of neurons during aging.

### Example 6

### Entorhinal Cortex Lesion as a Means of Evaluating Neuroregeneration

Lesion studies in which known connections are lesioned and changes in projection brain area are examined and used to explore structural plasticity in animal models. The impact of entorhinal cortex lesions on the function of the hippocampal loop is widely studied. To test for the effect of modulators of the invention on neuroregeneration, entorhinal cortex lesion is performed on mice, and modulators of neuroregeneration (C3aR or C5aR agonists or IGFBP-4, ULK, or ETBR antagonists) are administered to induce neuroregeneration. Unilateral entorhinal cortex lesion is performed as described previously (Stone et al., J. Neurosci. 18:3180-3185, 1998). After 4 or 14 days, the mice are perfused transcardially with 4% paraformaldehyde (for immunohistochemistry) or 3% glutaraldehyde and 2% paraformaldehyde (for electron microscopy) and the brains are examined for neuroregeneration or neurogenesis by determining the 1) extension of astrocytic processes, 2) dye filling of astrocytes, 3) quantification of newly proliferation cells, and 4) quantification of newly formed neurons (set out in Examples 8-11 below).

### Example 7

### Cell Culture

Primary cultures, enriched in reactive astrocytes, are prepared from 2-day old mice as described by Pekny et al. (Exp. Cell. Res. 239:332-343, 1998). Cell cultures are prepared from 1) normal mice, 2) normal mice treated with a modulator of neuroregeneration (an agent identified as an agonist of C3aR or C5aR, or an antagonist of IGFBP-4, ULK, or ETBR), and optionally, 3) GFAP−/−Vim−/− mice. Cultures are grown for 10 days in 96-well plates (Greiner Bio-One, Frickenhausen, Germany) containing Dulbecco's modified Eagle's medium (D5671 Sigma-Aldrich, St Louis, Mo., USA), 10% fetal calf serum (Gibco BRL, Paisly, UK), L-glutamine (2 mM), and penicillin/streptomycin (Gibco BRL). Cultures are then evaluated for neurite outgrowth and extension of astrocytic process. Agents which promote neurite outgrowth and inhibit the extension of astrocytic processes are selected.

### Example 8

### Evaluation of Astrocytic Processes by Immunohistochemistry

Brains are fixed for 1 day at 4° C. in paraformaldehyde and immersed in 30% sucrose and 0.05M sodium phosphate, pH 7.3, for 3 days at 4° C. Horizontal cryosections (25 μm) are made covering the same area of hippocampus as analyzed by electron microscopy and by Ki67/BrdU immunostaining (see below), and stored in a cryoprotectant (50% 0.05M sodium phosphate, pH 7.3, 30% ethylene glycol, 20% glycerol) at −20° C. After several washes in PBS, the sections are incubated in 0.05% glycine in PBS for 1 h at room temperature and permeabilized overnight in PBS containing 0.5% Tween 20 and 1% BSA at 4° C. Then, the sections are incubated with monoclonal antibody against glutamine synthase (Chemicon International; 1:100) or rabbit anti-cow S100 (DAKO, 1:200) in 0.01% Tween 20 and 1% BSA in PBS overnight. Following day, goat anti-mouse antibodies conjugated with Alexa 488 (Molecular Probes, Eugene, Oreg., USA; 1:500) or goat anti-rabbit antibodies conjugated with Alexa 568 (Molecular Probes, 1:500) are incubated with the sections overnight at 4° C. For nuclear staining, propidium iodide (Sigma-Aldrich) is added to the last wash before mounting. Astrocytes and their processes are quantified on superimposed pictures of 9 serial confocal images covering a thickness of 8 μm (glutamine synthase detection) or 4 serial confocal images covering a thickness of 3 μm (S100 detection) obtained with a laser-scanning confocal microscope (TCS NT, Leica, Heidelberg, Germany) and a 40× objective. Astrocytes with clearly visible cell nuclei and soma are selected. The length of the longest cellular processes of 20 such astrocytes in the molecular layer in the dentate gyrus of the hippocampus in each mouse are measured. At day 4 after entorhinal cortex lesion, mice are examined using glutamine synthase immunostaining. Mice are also examined using S100 immunostaining both at day 4 and 14. Astrocytic processes are compared in 1) normal mice, 2) normal mice treated with a modulator of neuroregeneration (an agent identified as an agonist of C3aR or C5aR, or an antagonist of IGFBP-4, ULK, or ETBR), and optionally, 3) GFAP−/−Vim−/− mice. Agents which inhibit astrocytic process length are selected.

### Example 9

### Dye Filling of Astrocytes

To evaluate the effect of the modulators of C3aR, C5aR, IGFBP-4, ULK, and ETBR on hypertrophy of cellular processes of reactive astrocytes, dye-filling experiments are peformed, which allow 3D reconstruction of astrocytes in situ. Intracellular injection of astrocytes in lightly fixed tissue slices is performed as described (Bushong et al., Neurosci. 22:183-92, 2002; Bushong et al., J. Comp. Neurol. 462:241-51, 2003). On day 4 after entorhinal cortex lesion, the mice are transcardially perfused with oxygenated Ringer's solution (0.79% NaCl, 0.038% KCl, 0.02% MgCl2.6H₂O, 0.018% Na 2HPO₄, 0.125% NaHCO3, 0.03% CaCl₂.2H₂O, 0.2% dextrose, 0.02% xylocalne), followed by 4% paraformaldehyde in PBS (pH 7.4,) for 8-10 minutes, both at 37° C. The brain is placed in ice-cold PBS and cut with a vibratome into 75 μm horizontal slices. The slices are stored in PBS at 4° C. and examined with an Olympus BX50WI microscope using infrared-DIC optics (Olympus, Melville, N.Y., 60× water objective NA 1.4). Astrocytes in the medial outer molecular layer of the dentate gyrus of the hippocampus are identified by the shape and size of their somata. Glass micropipettes (OD 1.00 mm, ID 0.58 mm) are pulled on a vertical puller (David Kopf Instruments, Tujunga, Calif.) and backfilled with 5% aqueous lucifer yellow (Sigma-Aldrich). Astrocytes are impaled and iontophoretically injected with the dye using 1-second pulses of negative current (0.5 Hz) for 1-2 minutes. After several cells are filled, the slices are placed in ice-cold 4% paraformaldehyde for at least 1 hour. For immunolabelling of GFAP in the dye-filled astrocytes, the slices are repeatedly washed in PBS and permeabilized for 1 h at room temperature in PBS containing 1% BSA, 0.25% Triton X-100 and 3% normal donkey serum, followed by incubation with guinea pig antibodies against GFAP (Sigma-Aldrich, 1:100) for 48 h at 4° C. in PBS containing 1% BSA, 0.1% Triton X-100 and 0.3% normal donkey serum. After washing several times in PBS, donkey anti-guinea pig antibodies conjugated with RRX (Jackson ImmunoResearch, West Groove, Pa., 1:300) are added to the slices, incubated overnight at 4° C. and then mounted in gelvatol (Harlow and Lane, 1988). The slices are examined using a Radiance2000 laser scanning confocal system (Bio-Rad, Hercules, Calif.) attached to a Nikon E600FN microscope (Kanagawa, Tokyo, Japan) with a 60× oil immersion objective (NA 1.4). Image visualization and analysis are performed using Imaris 3.3 (Bitplane, Zurich, Switzerland) and ImageJ (NIH, Bethesda, Md.) software. Quantification of neuropil volume reached by a dye-filled astrocyte is performed on 3D reconstructed cells in 3 wild-type and 3 GFAP−/−Vim−/− mice (in total 57 cells quantified) using Imaris 3.3 software. The number and character of cell processes reaching outside a 40 μm wide circle, centered around the soma, are assessed by using ImageJ software on superimposed serial images in of 61 dye-filled astrocytes. Astrocytic processes are compared in 1) normal mice, 2) normal mice treated with a modulator of neuroregeneration (an agent identified as an agonist of C3aR or C5aR, or an antagonist of IGFBP-4, ULK, or ETBR), and 3) GFAP−/−Vim−/− mice. Agents which inhibit extension of astrocytic processes are selected.

### Example 10

### Quantification of Proliferating Cells in the Brain

Brains are immersion-fixed for 1 day in 4% buffered paraformaldehyde and paraffin-embedded. Horizontal microtome sections (8 μm) are taken between 2000 and 2500 μm below the upper surface of the hippocampus, which is the region showing most prominent signs of neurodegeneration and astrocyte activation after entorhinal cortex lesion. Sections are rehydrated and antigen-retrieved with 0.01 M citrate buffer, pH 6.0, in a microwave oven for 15 min. After incubation in PBS containing 1% BSA and 0.05% Triton X-100 for 30 min to block nonspecific background activity, the sections are incubated first with rat Ki67 antibodies (clone TEC-3, DAKO) diluted 1:25 in PBS containing 0.1% BSA and 0.05% Triton X-100, and then with rabbit anti-cow S100 (DAKO) diluted 1:300, both for 1 h. After incubation with lectin buffer (PBS, pH 6.8, containing 1% Triton X-100, 0.1 mM MgCl₂, 0.1 mM MnCl₂, and 0.1 mM CaCl₂) for 1 h, the sections are incubated with biotinylated lectin from Bandeiraea simplicifolia (isolectin, Sigma-Aldrich), diluted 1:10 in lectin buffer, for 1 h. The secondary antibodies are Cy3-conjugated anti-rat (Jackson Immuno Research, West Grove, Pa., USA), Alexa 488-conjugated anti-rabbit, and Alexa 633-conjugated streptavidin (both Molecular Probes). All incubations are performed at room temperature, and sections are rinsed in PBS after each step. A laser-scanning confocal microscope and software (TCS NT, Leica) with a 63× objective is used to examine Ki67-positive (Ki67^{pos}) cells in the dentate gyrus on the injured side of the hippocampus and to assess their positivity for S100 and isolectin on 8 optical sections covering the thickness of 8 μm of the whole dentate gyrus of the hippocampus. Quantification of Ki67^{pos} cells in the subventricular zone (SVZ) is made in four consecutive 8 μm sections per mouse using an epifluoresence microscope (Nikon Eclipse E1000, Nikon Instruments Europe B.V., Badhoevedorp, The Netherlands). Cell proliferation is compared in 1) normal mice, 2) normal mice treated with a modulator of neuroregeneration (an agent identified as an agonist of C3aR or C5aR, or an antagonist of IGFBP-4, ULK, or ETBR), and 3) GFAP−/−Vim−/− mice. Agents which promote cell proliferation are selected

### Example 11

### Quantification of Newly Generated Neurons in the Brain

Mice are injected intraperitoneally with 5-bromo-2′-deoxyuridine (BrdU, 300 mg/kg body weight, Sigma-Aldrich), twice daily from day one to day seven after entorhinal cortex lesion and perfused six days after the last injection transcardially with 4% paraformaldehyde. Brains are fixed for one day at 4° C. in paraformaldehyde and horizontal vibratome sections (50 μm) are obtained as set out above. Mouse anti-NeuN antibody (Chemicon International), diluted 1:100, followed by Alexa 568-conjugated anti-mouse (Molecular Probes), diluted 1:500 are used for immunohistochemical staining. The sections are processed as described above (in Quantification of astrocytic processes). To unmask the BrdU antigen, the sections are incubated in 2N HCl for 2 h, and then washed several times in PBS, before incubation with FITC-conjugated rat anti-BrdU antibody (clone BU1/75, Accurate Chemicals, Westbury, N.Y., USA), diluted 1:100. A laser-scanning confocal microscope and software (TCS NT, Leica) is used to quantify the number of BrdU^{pos} and BrdU^{pos}NeuN^{pos} cells in the granule cell layer of the dentate gyrus of the hippocampus on the injured side, on 25 optical sections covering the thickness of 50 μm and the whole granule cell layer of the dentate gyrus of the hippocampus. Cell number is compared in 1) normal mice, 2) normal mice treated with a modulator of neuroregeneration (an agent identified as an agonist of C3aR or C5aR, or an antagonist of IGFBP-4, ULK, or ETBR), and 3) GFAP−/−Vim−/− mice. Agents which promote generation of new neurons (neurogenesis or neuroregeneration) are identified.

### Example 12

### Methods of Evaluating Cognitive Function in Mice

To evaluate improvement in cognitive function with a modulator of neuroregeneration, mice are inflicted with neural injury, e.g., middle cerebral artery occlusion (MCAO), electrical injury, and entorhinal cortex lesion. After infliction of neural injury, animals are treated with one or more of the various modulators of neuroregeneration identified herein. A variety of tests are available to one of skill in the art for evaluating improvement in cognitive function. The three most common tests used for evaluating cognitive function in mice are the Morris water maze test, the active avoidance test, and the contextual fear conditioning test. These cognitive tests are described in detail in e.g. in Current Protocols in Neuroscience (ed. Crawley et al., © 1999 John Wiley & Sons, Inc.). Improvement in cognitive function is compared in 1) normal mice, 2) normal mice treated with a modulator of neuroregeneration (an agent identified as an agonist of C3aR or C5aR, or an antagonist of IGFBP-4, ULK, or ETBR), and 3) GFAP−/−Vim−/− mice. Agents which promote improvement in cognitive function are selected.

### Example 13

### Optic Nerve Injury and Treatment with Modulators of Neuroregeneration

Through their ability to become reactive and form scars after injury, mature astrocytes represent a key barrier to optic nerve regeneration in older mice. Suppression of glial scarring following injury is sufficient to allow long distance and rapid regeneration of the severed optic nerve and target innervation in mature mice.

Optic nerve injury is a standard model for studying CNS regeneration. Rodent retinal ganglion cells (RGCs), whose axons form the optic nerve pathway, normally do not have the ability to regenerate their axons through an injured optic nerve. When provided with permissive substrate or given a novel treatment, only a small population of severed axons are induced to regenerate slowly and terminate before they even reach the targets (Li et al., J. Neurosci. 23:7783-7788, 2003; Yin et al., J. Neurosci. 23:2284-2293, 2003).

To test for the effect of modulators of the invention on optic nerve regeneration, optic nerve crush is performed on mice and modulators of neuroregeneration (C3aR or C5aR agonists or IGFBP-4, ULK, or ETBR antagonists) are administered to induce neuroregeneration. In control mice, most axons degenerate and rapidly retract after injury. In treated mice, many severed axons survive, and axon extension is measured. Many regenerating axons develop structures resembling growth cones at their tips, indicating their strong ability to regenerate.

To quantify neuroregeneration, FluoroGold is placed in the spinal cords (SCs) of mice immediately after optic nerve crush to label RGCs whose axons connect or regenerate to the SC. RGCs with regenerated axons are counted in 1) normal mice, 2) normal mice treated with a modulator of neuroregeneration (an agent identified as an agonist of C3aR or C5aR, or an antagonist of IGFBP-4, ULK, or ETBR), and 3) GFAP−/−Vim−/− mice. The number of RGCs with regenerated axons are expressed as a percentage of RGCs in the uninjured retina. Agents which promote regenerated axons are selected.

### Example 14

### C3aR and C5aR Expression on Neural Stem Cells and Neural Progenitors

Immunocytochemical analysis of clonally derived neural stem cells from adult rat hippocampus showed universal expression of C3aR and C5aR. Both receptors appeared to be localized in the cell membrane and were homogeneously distributed. The specificity of the anti-C3aR and anti-C5aR antibody, respectively was confinned by the absence of immunostaining on brain sections from mice deficient in C3aR (Kildsgaard et al., J. Immunol. 165:5406-5409, 2000) and C5aR (Hopken et al., Nature 383: 86-89, 1996), respectively. As demonstrated by nestin positivity, the cells retained their stem cell phenotype.

Dcx is a marker of migrating neuroblasts, which are normally present in the SVZ and rostral migratory stream of adult brain (Nacher et al., Eur. J. Neurosci. 14:629-644, 2001). In both regions, all Dcx^{pos} cells were also positive for C5aR, and many were positive for C3aR. Both C3aR and C5aR appeared to be membrane bound and homogeneously distributed. Immunostaining for NeuN, a marker of differentiated neurons, showed that over 95% of NeuN^{pos} cells were also positive for the C3aR and C5aR. Thus, neural stem cells, migrating neuroblasts, and mature neurons express C3aR and C5aR.

### Example 15

### Reduced Basal Neurogenesis in Mice Treated with a C3aR Antagonist

To assess whether signaling through the C3aR plays a role in basal neurogenesis, wild-type C57BL/6 mice (8 week old male; n=12) were injected with a nonpeptide antagonist of the C3aR, SB290157 (Calbiochem, San Diego, Calif., USA; 500 μg/mouse; Ames et al., J. Immunol. 166:6341-6348, 2001) diluted in PBS and DMSO (1.16% v/v) twice daily or vehicle for 10 days. Control mice (n=10) were given PBS and DMSO (1.16% v/v). During the first 7 days, all the mice also received bromodeoxyuridine (BrdU) (Sigma-Aldrich; 200 mg/kg). On day 10 after the first injection, the mice were deeply anesthetized and perfused.

Mouse brains were embedded in paraffin, cut into 8-μm sections, and stained with hematoxylin and erytrosin. For immunohistochemical evaluation, the sections were deparaffinized, permeabilized in 0.01 M citric acid (pH 6.0), heated twice for 5 min each in a microwave oven for antigen retrieval, and blocked with 0.1% BSA and 0.05% Triton-X-100 in PBS. Negative control was performed by omitting the primary antibody, unless stated otherwise.

Newly formed neurons were visualized by staining with biotinylated mouse anti-NeuN monoclonal antibody (Chemicon, Temecula, Calif., USA; 1:100) followed by Cy3-conjugated streptavidin (Sigma-Aldrich; 1:100) or Alexa633-conjugated streptavidin (Molecular Probes; 1:500) in combination with FITC-conjugated rat anti-BrdU antibody (Accurate Chemical, Westbury, N.Y., USA; 1:75). NeuN^{pos} BrdU^{pos} cells were counted in the penumbra on 2-3 sections per mouse. BrdU^{pos} cells were also counted in the SVZ. The sections were further stained with mouse anti-BrdU antibody (DAKO; 1:100) and Alexa 568 conjugated goat anti-mouse Ig (Molecular Probes; 1:500). BrdU^{pos} and NeuN^{pos}BrdU^{pos} cells were counted in one hemisphere on 4-12 sections 160 μm apart/region in the granular layer of the olfactory bulb (bregma 3.2 mm to 4.28 mm) and dentate gyrus subgranular zone (bregma −1.34 mm to −3.64 mm).

Migrating neural progenitor cells were detected with goat anti-doublecortin (Dcx) antibody (Santa Cruz Biotechnology, Santa Cruz, Calif., USA; 1:50) followed by Alexa488-conjugated donkey anti-goat Ig (Molecular Probes; 1:100). Sections were further stained with mouse anti-BrdU antibody. BrdU^{pos} and Dcx^{pos}BrdU^{pos} cells were counted in one hemisphere on 4-12 sections 160 μm apart/region in the SVZ (bregma 0.14 mm to 1.1 mm), the granular layer of the olfactory bulb (bregma 3.2 mm to 4.28 mm), and dentate gyrus subgranular zone (bregma −1.34 mm to −3.64 mm).

Proliferating migrating neuroblasts (Dcx^{pos}BrdU^{pos}), newly formed neurons (NeuN^{pos}BrdU^{pos}) and BrdU^{pos} cells were counted in the two principal sites of adult neurogenesis, SVZ and the dentate gyrus subgranular zone of the hippocampus, as well as in the olfactory bulb, the final destination for the neuroblasts originating in SVZ under basal conditions. In all three regions, the number of Dcx^{pos}BrdU^{pos} cells was reduced in mice that received the C3aR antagonist. This decrease was most pronounced in the olfactory bulb and dentate gyrus subgranular zone, where the number of Dcx^{pos}BrdU^{pos} cells was 34%, respectively 27% lower compared to control mice (P<0.0005) and was relatively mild in the SVZ (12.5% lower, P<0.05). Also, the fraction of these cells among the BrdU^{pos} cells was reduced in the C3aR antagonist-treated mice: 19.0±1.1 vs. 26.7±1.7%, P<0.005 in the olfactory bulb; 13.1±0.6 vs. 17.3±0.8%, P<0.0005 in the dentate gyrus subgranular zone; and 26.4±0.8 vs. 31.4±1.2%, P<0.005 in the SVZ. The quantification of NeuN^{pos}BrdU^{pos} cells in the olfactory bulb and dentate gyrus subgranular zone showed a 22-25% reduction in the number of these cells in the C3aR antagonist treated mice (P<0.005). The fraction of these cells among the BrdU^{pos} cells was reduced in the C3aR antagonist treated mice: 1.2±0.1 vs. 1.7±0.1%, P<0.005 in the olfactory bulb; 1.7±0.1 vs. 2.3±0.2%, P<0.05 in the dentate gyrus subgranular zone. The number of BrdU^{pos} cells in any of the regions was not affected by the C3aR antagonist treatment. These experimental results indicate that signaling through the C3aR positively regulates basal neurogenesis.

### Example 16

### Reduced Number of Neuroblasts in the SVZ in the Injured Hemisphere of C3−/− Mice after Brain Ischemia

To determine if complement activation plays a role in ischemia-triggered neurogenesis, C3−/− mice and wild-type controls were subjected to middle cerebral artery occlusion (MCAO) or transection (MCAT). Seven days after MCAO, control mice, but not the C3−/− mice, had 46% more Dcx^{pos} cells in the ipsilateral SVZ than in the contralateral SVZ (P<0.005). Moreover, the C3−/− mice had 24% fewer Dcx^{pos} cells in the ipsilateral SVZ than wild-type mice (P<0.05). Both groups had a similar number of these cells in the contralateral SVZ.

At both 7 and 21 d after MCAT, control mice had 38% more Dcx^{pos} cells in the ipsilateral SVZ than in the contralateral SVZ (P<0.01 and 0.05, respectively). In contrast, there was no significant difference between the numbers of Dcx^{pos} cells in the ipsilateral and contralateral SVZ of the C3−/− mice. These data indicate that the differentiation of neural stem cells into Dcx^{pos} neuroblasts in the SVZ is impaired in C3−/−mice.

### Example 17

### C3−/− and Control Mice Respond to Brain Ischemia by Increased Cell Proliferation in the Ipsilateral SVZ

To evaluate the proliferative capacity of cells in the SVZ, mice were injected with BrdU before and for 7 d after MCAT. In both C3−/− mice and wild-type control mice, BrdU^{pos} cells were more numerous in the ipsilateral than in the contralateral SVZ at 7 d (92.8±4.0 vs. 77.9±4.1, P<0.05 for C3−/− mice and 90.0±3.9 vs. 70.7±4.4, P<0.005 for wild-type mice) and 21 d after MCAT (14.5±1.0 vs. 10.6±0.8, P<0.01 and 16.3±1.5 vs. 10.9±1.1, P<0.05). Immunostaining for Ki-67 showed no difference between the ipsilateral and contralateral SVZ in the number of proliferating cells in C3−/− or wild-type mice 7 d after MCAO (132.5±16.3 vs. 128.2±11.8, NS, and 150.1±23.9 vs. 154.9±20.6, NS).

### Example 18

### C3−/− Mice Have Fewer Proliferating Nonmicroglial/Nonendothelial Cells in the Penumbra

In accordance with the findings in the SVZ, Ki-67 immunostaining showed no difference in the number of proliferating cells in the penumbra of C3−/− and wild-type mice 7 d after MCAO (107±11.4 vs. 110±19.2/mm³). However, the fraction of proliferating nonmicroglial, nonendothelial cells (Ki-67^{pos} isolectin^{neg}), a cell population enriched in neural progenitor cells, was 30% lower in the C3−/− mice (11.0±1.25 vs. 15.5±1.31%, P<0.05) than in controls.

### Example 19

### C3−/− Mice Have Fewer Neural Progenitor Cells in the Penumbra and Infarct Area

To determine the number of neural progenitor cells in the penumbra and infarct area, brain sections were immunohistochemically stained for nestin, a marker of neural progenitor cells that is also expressed by reactive astrocytes (Lendahl et al., Cell 60:585-595, 1990), and GFAP. Compared with controls, C3−/− mice had 50% fewer nestin^{pos} GFAP^{neg} cells in the infarct area (3.6±0.97 vs. 8.3±1.76 cells/section, P<0.05) and in the penumbra (15.9±2.02 vs. 28.4±2.22 cells/section, P<0.005) 7 d after MCAO and 30% fewer nestin^{pos} GFAP^{neg} cells in the penumbra (11.3±0.2 vs. 16.1±0.5 cells/10 mm², P<0.00001) 7 d after MCAT. Furthermore, the fraction of nestin^{pos} GFAP^{neg} cells among the nestin^{pos} cells was reduced by 29% in the C3−/− mice (5.4±0.1 vs. 7.6±0.2%, P<0.00001). No nestin^{pos} cells were detected in the penumbra of either group of mice at 21 d.

### Example 20

### C3−/− Mice Have Fewer Newly Formed Neurons after Ischemia

To determine if the reduction in the number of neural progenitor cells and migrating neuroblasts leads to reduced formation of new neurons, combined immunostaining for NeuN and BrdU was used. C3−/− mice had fewer newly-formed neurons (BrdU^{pos} NeuN^{pos} cells) in the penumbra both at 7 d (7.5±0.4 vs. 10.0±10.5 cells/10 mm², P<0.001) and at 21 d after MCAT (9.7±0.6 vs. 13.3±0.9 cells/10 mm², P<0.005) than controls. Also, the fraction of these cells among the BrdU^{pos} cells was reduced in the C3−/− mice (3.4±0.2 vs. 4.76±0.3%, P<0.005 and 4.9±0.4 vs. 6.5±0.4%, P<0.05) compared to controls.

To determine whether the BrdU^{pos} NeuN^{pos} cells are undergoing ischemia-induced apoptosis, triple-label immunostaining was performed with antibodies against BrdU, NeuN, and activated caspase-3, the latter being expressed in association with delayed ischemic neuronal death (Namura et al., J. Neurosci. 18:3659-3668, 1998). No activated caspase-3^{pos} cells were detected in the infarct area or within the penumbra 21 d after MCAT; however, 7 d after MCAT, caspase-3 immunoreactivity was confined to the infarct and NeuN immunoreactivity was confined to the penumbra. No BrdU^{pos} NeuN^{pos} cells were positive for activated caspase-3, which indicates that the BrdU^{pos} NeuN^{pos} cells were not undergoing caspase-3 dependent apoptosis. No activated caspase-3^{pos} cells were detected in the SVZ in either of the groups. Thus, these experiments support a role for the complement system in promoting neuroregeneration after cerebral ischemia via signaling through C3 and the C3aR.

### Example 21

### C3−/− Mice Have Larger Infarcts

To evaluate the role of C3 in infarction, MCAO and MCAT were performed on C3−/− mice and brain tissue volume was measured after infarction. Results showed that cerebral infarct volume was 24% greater in C3−/− mice than in wild-type mice (16.3±0.31 vs. 13.1±1.07 mm3, P<0.01) 7 d after MCAO. Seven days after MCAT, the infarct volume did not significantly differ between the groups, although there was a tendency toward a greater volume in the C3−/− mice (2.5±0.5 vs. 1.6±0.3 mm³, P=0.14). However, 21 d after MCAT, the C3−/− mice had lost twice as much tissue as controls (2.3±0.2 vs. 1.1±0.2 mm³, P<0.0005).

### Example 22

### Activation of Astrocytes and Microglia at the Infarct Border is Similar in C3−/− and Control Mice

To assess whether the activation of astrocytes and microglia was different between C3−/− and control mice, activation of astrocytes and microglia was observed at 7 days after MCAO. Reactive astrocytes were identified by staining with polyclonal antibody against GFAP (DAKO; 1:100) and inflammatory cells by biotinylated lectin (Sigma-Aldrich; 1:10) followed by TRITC-conjugated swine anti-rabbit Ig and FITC-conjugated streptavidin (both from DAKO). Staining intensity was assessed by measuring the width of the band of positive cells at the infarct border.

Immunostaining for GFAP showed a massive presence of highly GFAP^{pos} astrocytes at the infarct border in both C3^{-/-} and wild-type mice. Similarly, staining for isolectin, a marker of microglia and endothelial cells, demonstrated a rim of activated microglial cells around the ischemic lesion and migration of these cells into the infarct area in both groups. The width of the GFAP^{pos} band was comparable in C3^{-/-} and wild-type mice (456±62.3 and 440±25.1 μm), as was the width of the isolectin^{pos} band (367±18.8 and 319±20.3 μm).

This study showed that reactive gliosis and infiltration with microglial cells was comparable in C3^{-/-} and wild-type mice. Thus, complement activation products, at least C3, do not appear to be critical for the recruitment of reactive astrocytes and microglia to the necrotic brain region.

### Example 23

### Effect of a C3aR Agonist and Antagonist on Neural Stem Cell Differentiation

It was previously known that it was possible to modulate cells, other than neurons, via C3aR. Accordingly, C3aR antagonists blocked modulation via the receptor and C3aR agonists stimulated the receptor. To evaluate the effect of C3aR agonists on neural stem cell differentiation in vitro, a neural stem cell differentiation assay was performed as set out below.

Neural stem cells, e.g., HCNA 94/GFPH, were cultured with a C3aR agonist (C3a) at a concentration of either 10 nM or 100 nM for three to ten days in the presence and absence of a known C3aR antagonist, SB290157 (Ames at al., J. Immunol. 166: 6341-6348, 2001), at a concentration of 5-10 μM, administered to the culture either prior to or at the same time as the C3aR agonist. The number of cells that differentiated into neurons was measured by immunostaining with antibodies against MAP2 (Sigma) and visualized using 4′,6-Diamidino-2-phenylindole (DAPI) staining. A compound is regarded as a stimulator of neural stem cell differentiation into neurons if it stimulates differentiation into neurons at a rate at least 25% greater than control cells (cultured in the presence of medium alone). The effect of the C3aR agonist on neural stem cell differentiation was not blocked by SB290157 (see Table 5 below). Thus, SB290157 was not effective in inhibited neuronal cell differentiation at the concentrations tested.

Therefore, this study showed that it is necessary to use a neuronal cell-line to test the effects of various C3aR agonists and antagonists in effecting neural stem cell differentiation. This conclusion is contrary to the prior belief that all C3a receptor modulation was the same and independent of cell-type.

TABLE 5
THE EFFECT OF A C3aR AGONIST (WITH AND WITHOUT A C3aR
ANTAGONIST) ON NEURAL STEM CELL DIFFERENTIATION

| | % of MAP2+ |
|---|---|
| | Cells Among All DAPI+ |
| Culture Conditions | Cells (Mean ± SEM) |
| Culture medium alone | 2.54 ± 0.12 |
| C3aR agonist 10 nM | 3.30 ± 0.19 |
| C3aR agonist 100 nM | 3.70 ± 16 |
| C3aR agonist 10 nM + SD290157 10 μM | 3.70 ± 0.15 |
| SD290157 5 μM | 2.70 ± 0.19 |

Numerous modifications and variations in the invention as set forth in the above illustrative examples are expected to occur to those skilled in the art. Consequently only such limitations as appear in the appended claims should be placed on the invention.

What is claimed is:

1. A method of screening for a Complement 3a Receptor (C3aR) agonist comprising the steps of:
(a) contacting a composition comprising a neuron or neuronal stem cell that expresses a C3aR polypeptide with a test agent;
(b) measuring C3aR activation in the presence and absence of the test agent; and
(c) selecting as an agonist a test agent that induces C3aR activation.

2. A method according to claim 1, wherein the measuring step comprises the steps of:
(i) measuring binding between the test agent and the C3aR polypeptide; and measuring C3aR internalization, wherein a test agent that binds C3aR and induces C3aR internalization is identified as a C3aR agonist; or
(ii) measuring binding between the test agent and the C3aR polypeptide; and measuring C3aR signaling, wherein a test agent that binds C3aR and induces C3aR intracellular signaling is identified as a C3aR agonist.

3. A method according to claim 2, wherein the contacting step comprises contacting the C3aR polypeptide with a composition comprising a C3a polypeptide, and wherein binding between the test agent and the C3aR is measured by comparing C3a binding to C3aR in the presence and absence of the test agent, wherein decreased C3a binding to C3aR in the presence of the test agent indicates binding between the test agent and C3aR.

4. A method according to claim 1, wherein the measuring step comprises measuring cell growth, survival, or differentiation in the presence and absence of the test agent, wherein C3aR activity promotes cell growth, survival, or differentiation.

5. A method according to claim 4, wherein the measuring step comprises measuring differentiation in a neuronal stem cell.

6. A method of screening for a C3a receptor (C3aR) agonist, comprising steps of:
(a) contacting a composition comprising a C3aR polypeptide with a composition comprising a C3a polypeptide in the presence and absence of a test agent;
(b) measuring and comparing C3a binding to C3aR in the presence and absence of the test agent, wherein a test agent that inhibits C3a binding to C3aR is selected as a C3aR binding agent;
(c) contacting a C3aR binding agent of step (b) to a neuron or neuronal stem cell that expresses C3aR on its surface; and
(d) measuring and comparing receptor activation or receptor internalization in the presence and absence of the C3aR binding agent, wherein increased receptor activation or internalization in the presence of a C3aR binding agent compared to the absence identifies that C3aR binding agent as a C3aR agonist.

7. A method according to claim 1 or 6, wherein the cell is recombinantly modified to express elevated levels of C3aR on its surface.

8. A method according to claim 6, wherein binding between C3a and C3aR is detected by measuring a C3a-induced change to said cell.

9. A method according to claim 8, wherein the C3a-induced change in said cell is selected from the group consisting of a change in intracellular calcium ion concentration, a conversion of GTP to GDP, a change in cAMP concentration, cellular chemotaxis, and H₂O₂ production.

10. A method according to claim 1 or 6, wherein the C3aR polypeptide comprises an amino acid sequence at least 90% identical to a C3aR amino acid sequence selected from the group consisting of SEQ ID NOS: 5-7.

11. A method according to claim 6, wherein the C3a polypeptide comprises an amino acid sequence at least 90% identical to a C3a amino acid sequence selected from the group consisting of SEQ ID NOS: 2-3.

12. A method according to claim 1 or 6, further comprising steps of culturing a neuron or neural stem cell in the presence and absence of the C3aR agonist; measuring and comparing cell growth or survival or differentiation in the presence and absence of the C3aR agonist; and selecting a C3aR agonist that promotes increased survival or growth or differentiation of said neuron or neural stem cell.

13. A method according to claim 12, wherein the cell is selected from the group consisting of a hippocampal neuron or neural stem cell, a subventricular neuron or neuron stem cell, a cortical neuron or neuron stem cell, and a neuroblastoma cell.

14. A method according to claim 1 or 6, further comprising a step of making a C3aR agonist composition comprising the C3aR agonist and a pharmaceutically acceptable carrier.

15. A method according to claim 14, further comprising administering the C3aR agonist composition to a mammalian subject, and screening for neurological effects of the C3aR agonist on the subject.

16. A method according to claim 15, wherein the mammalian subject suffers from a neurological trauma, and wherein the mammalian subject is screened for neurological regeneration at a trauma site.

17. A method according to claim 16, wherein the mammalian subject suffers from neurological degeneration, and wherein the mammalian subject is screened for inhibition of the degeneration.

18. The method of claim 14, wherein the composition includes at least one additional factor which promotes neurogenesis or neuroregeneration selected from the group consisting of: NGF, BDNF, NT-3, 4, 5, or 6, CNTF, IGFI, IGFII, GDNF, GPA, bFGF, TGFβ, and apolipoprotein E.
